(19) United States
(12) Patent Application Publication
(10) Pub. No.: US 2026/0263167 A1
(43) Pub. Date: Sep. 10, 2026

(54) METHODS AND SYSTEMS FOR TUNNEL PLANNING AND NAVIGATION

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: João Pedro DE ALMEIDA BARRETO, Coimbra (PT); Nicholas R. LABRIOLA, Medway, MA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/666,436

(22) Filed: May 4, 2026

Related U.S. Application Data
(63) Continuation of application No. PCT/US2024/058596, filed on Dec. 5, 2024.
(60) Provisional application No. 63/606,662, filed on Dec. 6, 2023.

(51) Int. Cl. A61B 34/20; A61B 17/17; A61B 17/32; A61B 34/10

(52) U.S. Cl. CPC A61B 34/20; A61B 17/1764; A61B 2017/320056; A61B 2034/105; A61B 2034/107

(57) ABSTRACT

Some examples are directed to a method for displaying, by a surgical controller on a display device, surgical guidance information based on at least one calculated value indicative of a compatibility, for together supporting a replacement ligament, of a first tunnel in a first bone of a joint and a candidate second tunnel in a second bone of the joint. Some examples are directed to a surgical controller configured to display the surgical guidance information. Some examples are directed to a method for displaying, by a surgical controller on a display device, surgical guidance information based on at least one calculated value indicative of a risk of convergence of a first tunnel in a first bone and a candidate second tunnel in the first bone. Some examples are directed to a surgical controller configured to display the surgical guidance information.

### CROSS-REFERENCE TO RELATED APPLICATIONS

This application is a continuation of and claims the benefit of International Application No. PCT/US2024/058596 filed December 5, 2024. PCT/US2024/058596 claims priority to U.S. Provisional Patent Application Serial No. 63/606,662 filed on December 6, 2023 and titled “Planning and Navigation for ACL Tunnel Navigation (Tibia).” The applications referenced above are incorporated by reference herein in their entirety.

### BACKGROUND

The anterior cruciate ligament (ACL) serves as the primary mechanical restraint in the knee to resist anterior translation of the tibia relative to the femur. Similarly, the posterior cruciate ligament (PCL) serves as a mechanical restraint to resist posterior translation of the tibia relative to the femur. These cruciate ligaments contribute significantly to knee stability, and ACL injury is quite common. Most ACL injuries are complete tears of the ligament.

As ACL injuries occur often in patients that are young and active, reconstruction of the ACL is performed to enable a return to activity. The goal is to restore stability of the knee and reduce the chances of further damage to the meniscus and articular cartilage that may lead to degenerative osteoarthritis. Reconstruction may consist of placement of a substitute graft (e.g., autograft from either the central third of the patellar tendon or the hamstring tendons). The ends of the graft are placed into respective tunnels prepared through the femur and the tibia. The ends of the graft may be attached using interference screws or a suspensory fixation device like the ENDOBUTTON™ brand fixation devices manufactured by Smith & Nephew of Andover, Massachusetts, USA.

One challenge in ACL reconstruction is where the tunnels should be placed. The native ACL consists of 2 major bundles – the anteromedial (AM) and the posterolateral (PL) bundle. Often, the goal of the surgery is to place the reconstruction in an anatomical location, for example, placing a single tunnel within the footprint of the native ACL attachment site. In other cases, reconstruction may involve creating two tunnels in one or both of the femur and the tibia in an attempt to recreate the two native bundles.

There is considerable variability in the placement of tunnels relative to the planned-tunnel locations. It has been shown that relative to the planned-tunnel location, the errors in actual-tunnel location may vary from 8.3 to 13.9 millimeters (mm). Further, the failure rate in ACL reconstructions ranges from 10-15%, with 61% of the failures attributable to technical errors. Some 80% of the technical failures are femoral tunnel malposition and 37% are tibial tunnel malposition.

Patent Cooperation Treaty (PCT) Application Publication No. WO 2023/034194 to Quist et al., entitled “Methods and Systems of Ligament Repair,” the contents of which are incorporated herein by reference in their entirety, discloses methods and systems for verifying registrations of three-dimensional bone models with bone visible through an endoscope during ligament repair, as well as methods and systems that make use of such registered three-dimensional bone models to facilitate changes to bone tunnel plans for ligament repair. As explained by Quist et al., for various reasons a surgeon may elect not to use a planned-tunnel path, and thus elect not to use the planned entry location, exit location, or both, of the planned-tunnel path. Regardless of the reason for the election to change the tunnel path, in examples described by Quist et al. the surgeon is enabled to intraoperatively select a revised-tunnel entry, a revised-tunnel exit (if needed), and thus a revised-tunnel path through the bone.

For surgical procedures requiring a replacement ligament to extend from one bone of a joint to another, such as in the case of ACL reconstruction, a particular ligament graft may be harvested with a particular length. This graft length may be estimated based in part on the lengths of planned-tunnel paths through each of the bones and the distance between the openings out of each bone of the planned-tunnel paths through which the graft will pass, this latter distance corresponding generally to the length of the native ACL itself. During the surgical procedure itself, however, should the surgeon wish to deviate from one or more of the planned-tunnel paths for any of the bones, doing so could cause a change in the total distance that the ligament graft must traverse.

Furthermore, during a surgical procedure, it may be desirable to form more than one bone tunnel in a single bone. For example, it may be desirable to additionally conduct a meniscal root repair (MRR) when conducting the tibial portion of an ACL repair. An MRR may involve forming one or more bone tunnels through the tibia to each receive a respective suture that, in turn, is used to draw and bind a torn meniscal root back against the tibia. Even if an MRR is not being done, a surgeon may elect to perform ACL reconstruction by creating two tunnels in the femur to each accommodate a respective one of the ends of the AM and PL substitute ligament graft bundles, and may similarly involve creating two tunnels in the tibia to each accommodate the other ends of the AM and PL substitute ligament graft bundles.

### SUMMARY

One example is a method comprising storing, by a surgical controller, data defining a first tunnel in a first bone of a joint, the first tunnel having a first longitudinal axis; tracking, by the surgical controller, a drill axis of an instrument with respect to a second bone of the joint; defining, by the surgical controller, a candidate second tunnel in the second bone having a second longitudinal axis corresponding to the drill axis of the instrument; calculating, by the surgical controller based at least on the candidate second tunnel and the data defining the first tunnel, at least one value indicative of a compatibility of the candidate second tunnel with the first tunnel for together supporting a replacement ligament; and displaying, by the surgical controller on a first display device, surgical guidance information based on the at least one value indicative of the compatibility.

In the example method, calculating the at least one value indicative of the compatibility of the candidate second tunnel with the first tunnel may comprise: determining a first length based on or obtained from the data defining the first tunnel, the first length being the length of the first tunnel in the first bone; calculating a second length, the second length being the length of the candidate second tunnel in the second bone; calculating a third length, the third length being a distance between openings of the first tunnel in the first bone and the candidate second tunnel in the second bone; calculating, as a candidate traversal distance, a sum of the first length, the second length, and the third length; and determining, as the at least one value indicative of the compatibility, an amount of difference between the candidate traversal distance and a length of the replacement ligament.

In the example method, the surgical guidance information displayed on the first display device may further comprise a length value corresponding to the amount of difference between the candidate traversal distance and the length of the replacement ligament.

In the example method, the displaying, by the surgical controller on the first display device, the surgical guidance information, may further comprise displaying an alert responsive to the candidate traversal distance being greater than the length of the replacement ligament; and removing a previously-displayed alert responsive to the candidate traversal distance being less than or equal to the length of the replacement ligament.

The example method may further comprise, during the tracking, displaying, by the surgical controller on the first display device, a representation of the candidate second tunnel in association with video frames of the second bone, wherein the alert comprises a change in appearance of the representation.

In the example method, the change in appearance of the representation may comprise at least one of a change in color of the representation, and a change in opacity of the representation.

In the example method, calculating the at least one value indicative of the compatibility of the candidate second tunnel with the first tunnel may comprise generating, based at least on openings into the joint of the first tunnel in the first bone and the candidate second tunnel in the second bone, a candidate path of the replacement ligament through the joint; and determining, as the at least one value indicative of the compatibility, an amount of impingement of the candidate path by at least one of the first bone and the second bone.

In the example method, the surgical guidance information displayed on the first display device may further comprise a percentage amount corresponding to the amount of impingement of the candidate path.

In the example method, the displaying, by the surgical controller on the first display device, the surgical guidance information may further comprise displaying an alert responsive to the amount of impingement being greater than or equal to a threshold level of impingement; and removing a previously-displayed alert responsive to the impingement being less than the threshold level of impingement.

The example method may further comprise, during the tracking, displaying, by the surgical controller on the first display device, a representation of the candidate second tunnel in association with video frames of the second bone, wherein the alert comprises a change in appearance of the representation.

In the example method, the change in appearance of the representation may comprise at least one of a change in color of the representation, and a change in opacity of the representation.

In the example method, the instrument may be an adjustable aimer and the tracking, by the surgical controller, of the drill axis of the instrument with respect to the second bone of the joint may comprise tracking a pose of the adjustable aimer with respect to the second bone; receiving an aimer angle of the adjustable aimer; and based on a predetermined geometry of the adjustable aimer, the pose of the adjustable aimer and the aimer angle, calculating the drill axis of the adjustable aimer with respect to the second bone.

The example method may comprise receiving, by the surgical controller, a user selection of the predetermined geometry of the adjustable aimer.

The example method may comprise processing, by the surgical controller, a machine-readable marker associated with the adjustable aimer to determine an identification of the adjustable aimer; and retrieving, by the surgical controller, the predetermined geometry based on the identification.

In the example method, receiving the aimer angle of the adjustable aimer may further comprise processing, by the surgical controller, video frames of the adjustable aimer to determine the aimer angle.

In the example method, a 3D bone model may be registered to the second bone.

The example method may further comprise displaying, by the surgical controller on the first display device and/or a second display device, a visual representation of the 3D bone model; and displaying, by the surgical controller on the first display device and/or the second display device, at least the candidate second tunnel with respect to the visual representation of the 3D bone model.

In the example method, the displaying of the candidate second tunnel with respect to the visual representation of the 3D bone model may be conducted by the surgical controller responsive to the surgical controller receiving, during the tracking, a selection by a user of a current pose of the instrument.

Another example is a surgical controller comprising: at least one processor configured to couple to at least a first display device; a memory coupled to the at least one processor, the memory storing instructions that, when executed by the at least one processor, cause the at least one processor to: store data defining a first tunnel in a first bone of a joint, the first tunnel having a first longitudinal axis; track a drill axis of an instrument with respect to a second bone of the joint; define a candidate second tunnel in the second bone having a second longitudinal axis corresponding to the drill axis of the instrument; calculate, based at least on the candidate second tunnel and the data defining the first tunnel, at least one value indicative of a compatibility of the candidate second tunnel with the first tunnel for together supporting a replacement ligament; and display, on the first display device, surgical guidance information based on the at least one value indicative of the compatibility.

In the example surgical controller, to calculate the at least one value indicative of the compatibility of the candidate second tunnel with the first tunnel, the instructions may cause the at least one processor to: determine a first length based on or obtained from the data defining the first tunnel, the first length being the length of the first tunnel in the first bone; calculate a second length, the second length being the length of the candidate second tunnel in the second bone; calculate a third length, the third length being a distance between openings of the first tunnel in the first bone and the candidate second tunnel in the second bone; calculate, as a candidate traversal distance, a sum of the first length, the second length, and the third length; and determine, as the at least one value indicative of the compatibility, an amount of difference between the candidate traversal distance and a length of the replacement ligament.

In the example surgical controller, the surgical guidance information displayed on the first display device may comprise: a length value corresponding to the amount of difference between the candidate traversal distance and the length of the replacement ligament.

In the example surgical controller, for displaying, on the first display device, the surgical guidance information, the instructions may cause the at least one processor to: display an alert responsive to the candidate traversal distance being greater than the length of the replacement ligament; and remove a previously-displayed alert responsive to the candidate traversal distance being less than or equal to the length of the replacement ligament.

In the example surgical controller, the instructions may cause the at least one processor to: during tracking of the drill axis, display, on the first display device, a representation of the candidate second tunnel in association with video frames of the second bone, wherein the alert comprises a change in appearance of the representation.

In the example surgical controller, the change in appearance of the representation may comprise at least one of a change in color of the representation, and a change in opacity of the representation.

In the example surgical controller, to calculate the at least one value indicative of the compatibility of the candidate second tunnel with the first tunnel, the instructions may cause the at least one processor to: generate, based at least on openings into the joint of the first tunnel in the first bone and the candidate second tunnel in the second bone, a candidate path of the replacement ligament through the joint; and determine, as the at least one value indicative of the compatibility, an amount of impingement of the candidate path by at least one of the first bone and the second bone.

In the example surgical controller, the surgical guidance information displayed on the first display device may comprise: a percentage amount corresponding to the amount of impingement of the candidate path.

In the example surgical controller, to display, on the first display device, the surgical guidance information, the instructions may cause the at least one processor to: display an alert responsive to the amount of impingement being greater than or equal to a threshold level of impingement; and remove a previously-displayed alert responsive to the impingement being less than the threshold level of impingement.

In the example surgical controller, the instructions may cause the at least one processor to: during tracking, display, on the first display device, a representation of the candidate second tunnel in association with video frames of the second bone, wherein the alert comprises a change in appearance of the representation.

In the example surgical controller, the change in appearance of the representation may comprise at least one of a change in color of the representation, and a change in opacity of the representation.

In the example surgical controller, the instrument may be an adjustable aimer and, to track of the drill axis of the instrument with respect to the second bone of the joint the instructions may cause the at least one processor to: track a pose of the adjustable aimer with respect to the second bone; receive an aimer angle of the adjustable aimer; and based on a predetermined geometry of the adjustable aimer, the pose of the adjustable aimer and the aimer angle, calculate the drill axis of the adjustable aimer with respect to the second bone.

In the example surgical controller, the instructions may cause the at least one processor to: receive, by the surgical controller, a user selection of the predetermined geometry of the adjustable aimer.

In the example surgical controller, the instructions may cause the at least one processor to: process a machine-readable marker associated with the adjustable aimer to determine an identification of the adjustable aimer; and retrieve the predetermined geometry based on the identification.

In the example surgical controller, to receive the aimer angle of the adjustable aimer the instructions may cause the at least one processor to: process video frames of the adjustable aimer to determine the aimer angle.

In the example surgical controller, a 3D bone model may be registered to the second bone.

In the example surgical controller, the instructions may cause the at least one processor to: display, on the first display device and/or a second display device, a visual representation of the 3D bone model; and display at least the candidate second tunnel with respect to the visual representation of the 3D bone model.

In the example surgical controller, the instructions for causing the at least one processor to display the candidate second tunnel with respect to the visual representation of the 3D bone model may be executed responsive to the surgical controller receiving, during tracking, a selection by a user of a current pose of the instrument.

Yet another example is a method comprising: storing, by a surgical controller, data defining a first tunnel in a first bone, the first tunnel having a first longitudinal axis; tracking, by the surgical controller, a drill axis of an instrument with respect to the first bone; defining, by the surgical controller, a candidate second tunnel in the first bone having a second longitudinal axis corresponding to the drill axis of the instrument; calculating, by the surgical controller based at least on the candidate second tunnel and the data defining the first tunnel, at least one value indicative of a risk of convergence in the first bone of the candidate second tunnel with the first tunnel; and displaying, by the surgical controller on a first display device, surgical guidance information based on the at least one value indicative of the risk of convergence.

In the example method, the calculating, by the surgical controller, the at least one value indicative of the risk of convergence of the candidate second tunnel with the first tunnel in the first bone may further comprise measuring based at least on the data defining the first tunnel, as the at least one value indicative of the risk of convergence, a distance between the closest points along the first tunnel and the candidate second tunnel within the first bone.

In the example method, the surgical guidance information displayed on the first display device may further comprise a bone bridge thickness amount corresponding to the distance between the closest points.

In the example method, the displaying, by the surgical controller on the first display device, the surgical guidance information may further comprise displaying an alert responsive to the distance between the closest points being below a threshold distance; and removing a previously-displayed alert responsive to the distance between the closest points being greater than or equal to the threshold distance.

The example method may further comprise: during the tracking, displaying, by the surgical controller on the first display device, a representation of the candidate second tunnel in association with video frames of the first bone, wherein the alert comprises a change in appearance of the representation.

In the example method, the change in appearance of the representation may comprise at least one of: a change in color of the representation, and a change in opacity of the representation.

The example method may further comprise displaying, by the surgical controller on the first display device, a representation of the first tunnel in association with the video frames of the first bone.

In the example method, a 3D bone model may be registered to the first bone.

The example method may further comprise displaying, by the surgical controller on the first display device and/or a second display device, a visual representation of the 3D bone model; and displaying, by the surgical controller on the first display device and/or the second display device, at least the first tunnel with respect to the visual representation of the 3D bone model.

The example method may further comprise displaying, by the surgical controller on the first display device and/or the second display device, the candidate second tunnel with respect to the visual representation of the 3D bone model.

In the example method, the displaying of the candidate second tunnel with respect to the visual representation of the 3D bone model may be conducted by the surgical controller responsive to the surgical controller receiving, during the tracking, a selection by a user of a current pose of the instrument.

Yet another example is a surgical controller comprising: at least one processor configured to couple to at least a first display device; a memory coupled to the at least one processor, the memory storing instructions that, when executed by the at least one processor, cause the at least one processor to: store data defining a first tunnel in a first bone, the first tunnel having a first longitudinal axis; track a drill axis of an instrument with respect to the first bone; define a candidate second tunnel in the first bone having a second longitudinal axis corresponding to the drill axis of the instrument; calculate, based at least on the candidate second tunnel and the data defining the first tunnel, at least one value indicative of a risk of convergence in the first bone of the candidate second tunnel with the first tunnel; and display, on the first display device, surgical guidance information based on the at least one value indicative of the risk of convergence.

In the example surgical controller, to calculate the at least one value indicative of the risk of convergence of the candidate second tunnel with the first tunnel in the first bone, the instructions may cause the at least one processor to: measure based at least on the data defining the first tunnel, as the at least one value indicative of the risk of convergence, a distance between the closest points along the first tunnel and the candidate second tunnel within the first bone.

In the example surgical controller, the surgical guidance information displayed on the first display device may comprise: a bone bridge thickness amount corresponding to the distance between the closest points.

In the example surgical controller, to display, on the first display device, the surgical guidance information, the instructions may cause the at least one processor to: display an alert responsive to the distance between the closest points being below a threshold distance; and remove a previously-displayed alert responsive to the distance between the closest points being greater than or equal to the threshold distance.

In the example surgical controller, the instructions may cause the at least one processor to: during tracking, display, on the first display device, a representation of the candidate second tunnel in association with video frames of the first bone, wherein the alert comprises a change in appearance of the representation.

In the example surgical controller, the change in appearance of the representation may comprise at least one of: a change in color of the representation, and a change in opacity of the representation.

In the example surgical controller, the instructions may cause the at least one processor to: display, on the first display device, a representation of the first tunnel in association with the video frames of the first bone.

In the example surgical controller, a 3D bone model may be registered to the first bone.

In the example surgical controller, the instructions may cause the at least one processor to: display, on the first display device and/or a second display device, a visual representation of the 3D bone model; and display at least the first tunnel with respect to the visual representation of the 3D bone model.

In the example surgical controller, the instructions may cause the at least one processor to: display, on the first display device and/or the second display device, the candidate second tunnel with respect to the visual representation of the 3D bone model.

In the example surgical controller, the instructions to cause the at least one processor to display the candidate second tunnel with respect to the visual representation of the 3D bone model may be executed responsive to the surgical controller receiving, during tracking, a selection by a user of a current pose of the instrument.

### BRIEF DESCRIPTION OF THE DRAWINGS

For a detailed description of example embodiments, reference will now be made to the accompanying drawings in which:

FIG. 1 shows an anterior or front elevation view of right knee, with the patella removed;

FIG. 2 shows a posterior or back elevation view of the right knee;

FIG. 3 shows a view of the tibia from above and looking down upon the tibial plateau;

FIG. 4 shows a surgical system, in accordance with at least some embodiments;

FIG. 5 shows a conceptual drawing of a surgical site with various objects within the surgical site tracked, in accordance with at least some embodiments;

FIG. 6 is a display screen of a tablet computer providing a plan summary of an ACL repair, in accordance with at least some embodiments;

FIG. 7 is a display screen of the tablet computer providing an example edit screen, in accordance with at least some embodiments;

FIG. 8 is a display screen of the tablet computer providing another example edit screen, in accordance with at least some embodiments;

FIG. 9 is a display screen of the tablet computer displayed responsive to the selection of a save plan option in the display screen of FIG. 6, in accordance with at least some embodiments;

FIG. 10 is a display screen of the tablet computer showing a three-dimensional model of the tibia pursuant to placement of a fiducial marker at a fixed location on the tibia, in accordance with at least some embodiments;

FIGS. 11A-11E are an example video displays showing computer guidance for placement of a pilot ACL tunnel, in accordance with at least some embodiments;

FIG. 12 is a display screen of the tablet computer providing tibial tunnel settings, measurements, and guidewire capture options based on tracking of the instrument as shown in FIGS. 11A-E, in accordance with at least some embodiments;

FIG. 13 is a display screen of the tablet computer providing a view of the three-dimensional model of the tibia, in accordance with at least some embodiments;

FIG. 14A is a display screen of the tablet computer providing a planned tibial tunnel overview based on a captured guidewire position that is considered by the surgeon to be within all constraints and useful for drilling, in accordance with at least some embodiments;

FIG. 14B is an example video display presented on a display device showing computer guidance for placement of an MRR tunnel, in accordance with at least some embodiments;

FIG. 15 is a display screen of the tablet computer providing computer guidance for placement of a meniscal root repair (MRR) tunnel, pursuant to a pose of guidewire, and thus the trajectory of the drill axis, having been captured, in accordance with at least some embodiments;

FIG. 16 is a display screen of the tablet computer providing computer guidance for placement of the MRR tunnel, and enabling selection of options, in accordance with at least some embodiments;

FIG. 17A is an example video display of a display device showing computer guidance for placement of an MRR tunnel, in accordance with at least some embodiments;

FIG. 17B is an example video display of a display device showing computer guidance for placement of an MRR tunnel, in accordance with at least some embodiments;

FIG. 18 is a display screen of the tablet computer providing a view of the three-dimensional model of the tibia, showing the ACL tunnel prior to capture of a guidewire position for a planned tunnel, in accordance with at least some embodiments;

FIG. 19A is a display screen of the tablet computer providing a view of the surgical parameters and a Capture guidewire position button, in accordance with at least some embodiments;

FIG. 19B is an example video display of a display device showing computer guidance for placement of an MRR tunnel that may be presented to the surgeon while the tablet computer is presenting the content shown in FIG. 19A, in accordance with at least some embodiments;

FIG. 20 is a display screen of the tablet computer providing a view of the three-dimensional model of the tibia, showing both the planned tunnel trajectory and the ACL tunnel positions in relation to one another, in accordance with at least some embodiments;

FIG. 21A is a display screen of the tablet computer providing surgical parameters regarding the final guidewire position captured, in accordance with at least some embodiments;

FIG. 21B is an example video display of a display device showing computer guidance for placement of the MRR tunnel that may be presented to the surgeon while the tablet computer is presenting the content shown in FIG. 21A, in accordance with at least some embodiments;

FIG. 22 is a display screen of the tablet computer providing a view of the three-dimensional model of the tibia, along with representations of each of the final ACL tunnel, the planned (or previous) MRR tunnel, and a tunnel that would be formed were the guidewire position recently captured selected by the surgeon as the new planned tunnel, in accordance with at least some embodiments;

FIG. 23 shows a method, in accordance with at least some embodiments;

FIG. 24 shows a method, in accordance with at least some embodiments; and

FIG. 25 shows a computer system in accordance with at least some embodiments.

### DEFINITIONS

Various terms are used to refer to particular system components. Different companies may refer to a component by different names – this document does not intend to distinguish between components that differ in name but not function. In the following discussion and in the claims, the terms “including” and “comprising” are used in an open-ended fashion, and thus should be interpreted to mean “including, but not limited to… .” Also, the term “couple” or “couples” is intended to mean either an indirect or direct connection. Thus, if a first device couples to a second device, that connection may be through a direct connection or through an indirect connection via other devices and connections.

“Receiving … a … location” shall mean receiving data indicative of location on a bone within a coordinate space (e.g., a coordinate space of a view of an endoscope). Thus, example systems and methods may “receive … a revised-tunnel entry location” being data indicative of a proposed location of a tunnel entry point within a three-dimensional coordinate space. Other example systems and methods may “receive … a plurality of locations on a bone” being data indicative locations of an outer surface of a bone as part of registering a bone to a three-dimensional bone model.

An endoscope having “a single optical path” through an endoscope shall mean that the endoscope is not a stereoscopic endoscope having two distinct optical paths separated by an interocular distance at the light collecting end of the endoscope. The fact that an endoscope has two or more optical members (e.g., glass rods, optical fibers) forming a single optical path shall not obviate the status as a single optical path.

“Throughbore” shall mean an aperture or passageway through an underlying device. However, the term “throughbore” shall not be read to imply any method of creation. Thus, a throughbore may be created in any suitable way, such as drilling, boring, laser drilling, or casting.

“Counterbore” shall mean an aperture or passageway into an underlying device. In cases in which the counterbore intersects another aperture (e.g., a throughbore), the counterbore may thus define an internal shoulder. However, the term “counterbore” shall not be read to imply any method of creation. A counterbore may be created in any suitable way, such as drilling, boring, laser drilling, or casting.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Various examples are directed to methods and systems of ligament reconstruction and repair. The ligament repair (e.g., anterior cruciate ligament (ACL) repair) may be performed arthroscopically and computer assisted. Some examples include

The various examples were developed in the context of ACL repair, and thus the discussion below is based on the developmental context. However, the techniques are applicable to many types of ligament repair, such as medial collateral ligament repair, lateral collateral ligament repair, and posterior cruciate ligament repair. Moreover, the various example methods and systems can also be used for planning and placing anchors to reattach soft tissue, such as reattaching the labrum of the hip, the shoulder, or the meniscal root. Thus, the description and developmental context shall not be read as a limitation of the applicability of the teachings. In order to orient the reader, the specification first turns a description of the knee.

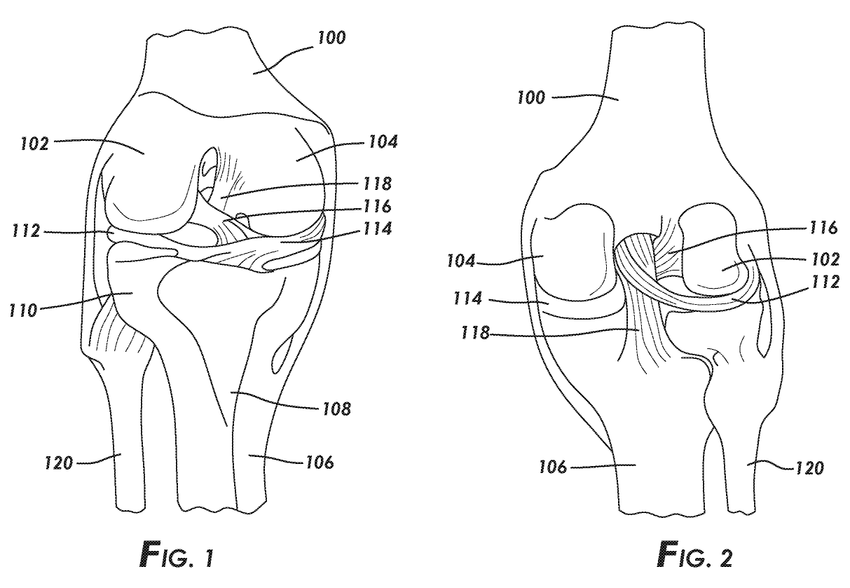

FIG. 1 shows an anterior or front elevation view of a right knee, with the patella removed. In particular, visible in FIG. 1 is lower portion of the femur 100 including the outer or lateral condyle 102 and the inner or medial condyle 104. The femur 100 and condyles 102 and 104 are in operational relationship to a tibia 106 including the tibial tuberosity 108 and Gerdy’s tubercle 110. Disposed between the femoral condyles 102 and 104 and the tibia 106 are the lateral meniscus 112 and the medial meniscus 114, each supported by and covering a respective portion of a tibial plateau 115. Several ligaments are also visible in the view of FIG. 1, such as the ACL 116 extending from the lateral side of femoral notch to the medial side of the tibia 106. Oppositely, the posterior cruciate ligament 118 extends from medial side of the femoral notch to the tibia 106. Also visible is the fibula 120, and several additional ligaments that are not specifically numbered.

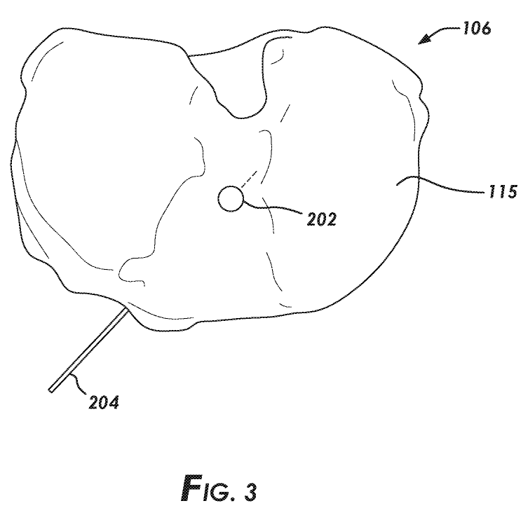

FIG. 2 shows a posterior or back elevation view of the right knee. In particular, visible in FIG. 2 is lower portion of the femur 100 including the lateral condyle 102 and the medial condyle 104. The femur 100 and femoral condyles 102 and 104 again are in operational relationship to the tibia 106, and disposed between the femoral condyles 102 and 104 and the tibia 106 are the lateral meniscus 112 and the medial meniscus 114 supported by and covering a respective portion of the tibial plateau 115. FIG. 2 further shows the ACL 116 extending from the lateral side of femoral notch to the medial side of the tibia 106, though the attachment point to the tibia 106 is not visible. The posterior cruciate ligament 118 extends from medial side of the femoral notch to the tibia 106, though the attachment point to the femur 100 not visible. Again, several additional ligaments are shown that are not specifically numbered.

The most frequent ACL injury is a complete tear of the ligament. Treatment involves reconstruction of the ACL by placement of a substitute graft (e.g., autograft from either the patellar tendon, quad tendon, or the hamstring tendons). The graft is placed into tunnels prepared within the femur 100 and the tibia 106. The current standard of care for ACL repair is to locate the tunnels such that the locations at which each of the femoral and tibial tunnels open into the joint is at the anatomical attachment location of the native ACL. Such tunnel placement at the attachment location of the native ACL attempts to recreate original knee kinematics.

As explained in Quist et al., in arthroscopic surgery, whereas it may be difficult to reach the attachment location of the native ACL to the femur 100 for tunnel formation, the location of the tunnel through the tibia 106 is relatively easy to reach. In either case, a drill wire may be used to create an initial tunnel or pilot hole. Once the surgeon verifies that the pilot hole is closely aligned with a planned-tunnel path through the bone, the bone tunnel is created by boring or reaming with another instrument (e.g., a reamer) that may use the drill wire as a guide.

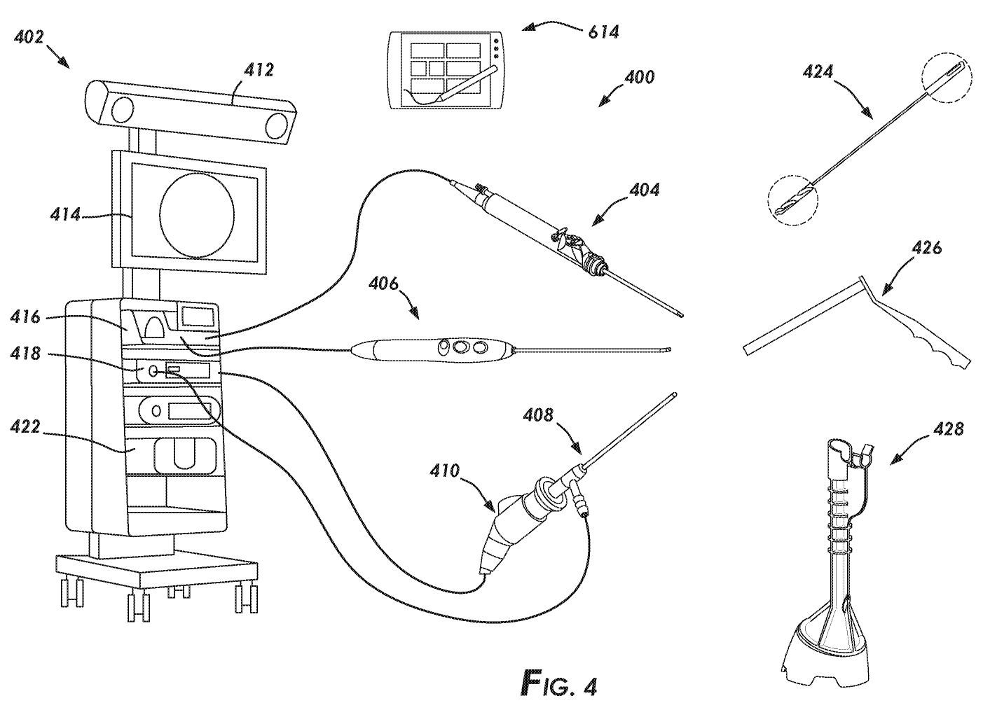

FIG. 3 shows a view of the tibia 106 from above and looking at the tibial plateau 115, showing a drill wire 204 used to create the initial tunnel or pilot hole between an anterior location (not shown) and a tunnel exit aperture 202.

Drilling of a tunnel may take place from either direction. In the case of a tibial tunnel, a surgeon generally has more flexibility in tunnel placement than that which is available for a femoral tunnel. Considering the tibial tunnel as an example, the tunnel may be drilled from a point on the anterior of the tibia 106 toward and through the tibial plateau 115, which is referred to as an “outside-in” procedure. Oppositely, the example tibial tunnel may be drilled from the inside of the joint into the tibial plateau 115 toward and to the anterior of the tibia 106, which is referred as an “inside-out” procedure. For the purpose of this disclosure, whether an “outside-in” or an “inside-out” procedure is to be used, the opening within the joint of the tibial tunnel will be referred to as the tunnel exit, and the opening at the anterior of the tibia will be referred to as the tunnel entry. For a tibial tunnel, computer-assisted navigation as described herein may be used to aid a surgeon with positioning of just the tunnel exit, while the tunnel entry - and thus the full tunnel trajectory - may be decided upon by the surgeon during the surgical procedure itself. According to this disclosure, because the surgeon may not necessarily have to adhere to an entire planned tunnel trajectory for the tibial tunnel, but may instead choose the tunnel entry more freely, surgical time may be reduced. Furthermore, according to this disclosure, computer-based surgical guidance may be made available to the surgeon to aid the surgeon with choosing the tunnel entry in a manner that satisfies various constraints, such as overall tunnel length constraints, bone bridge size constraints, and tunnel angle constraints.

The various examples discussed below are equally applicable to outside-in or inside-out procedures. The specification now turns to an example surgical system.

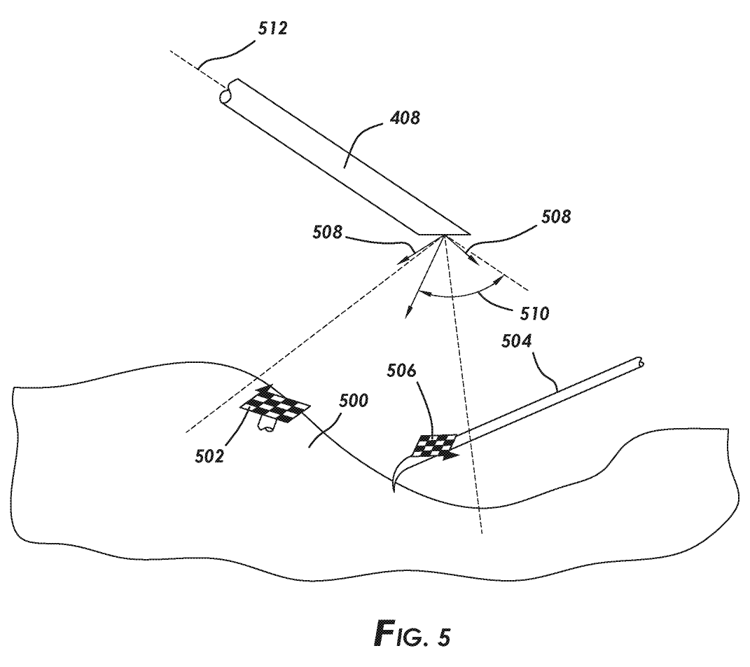

FIG. 4 shows a surgical system (not to scale) in accordance with at least some embodiments. In particular, the example surgical system 400 comprises a tower or device cart 402, an example mechanical resection instrument 404, an example plasma-based ablation instrument (hereafter just ablation instrument 406), and an endoscope in the example form of an arthroscope 408 and attached camera head 410. The arthroscope 408 defines a light connection or light post 420 to which light is provided, and the light is routed internally within the arthroscope 408 to illuminate a surgical field at the distal end of the arthroscope 408. The device cart 402 may comprise a camera 412 (illustratively shown as a stereoscopic camera), a display device 414, a resection controller 416, and a camera control unit (CCU) together with an endoscopic light source and surgical controller 418. In example cases the CCU and surgical controller 418 provides light to the light post 420 of the arthroscope 408, displays images received from the camera head 410. In example cases, the CCU and surgical controller 418 also implements various additional aspects, such as calibration of the arthroscope and camera head, displaying planned-tunnel paths on the display device 414, receiving revised-tunnel entry locations, calculating revised-tunnel paths, and calculating and displaying various parameters that show the relationship between the revised-tunnel path and the planned-tunnel path. Thus, the CCU and video controller is hereafter referred to as surgical controller 418. In other cases, however, the CCU and video controller may be a separate and distinct system from the controller that handles aspects of intraoperative changes, yet the separate devices would nevertheless be operationally coupled.

The example device cart 402 further includes a pump controller 422 (e.g., single or dual peristaltic pump). Fluidic connections of the mechanical resection instrument 404 and ablation instrument 406 are not shown so as not to unduly complicate the figure. Similarly, fluidic connections between the pump controller 422 and the patient are not shown so as not to unduly complicate the figure. In the example system, both the mechanical resection instrument 404 and the ablation instrument 406 are coupled to the resection controller 416 being a dual-function controller. In other cases, however, there may be a mechanical resection controller separate and distinct from an ablation controller. The example devices and controllers associated with the device cart 402 are merely examples, and other examples include vacuum pumps, patient-positioning systems, robotic arms holding various instruments, ultrasonic cutting devices and related controllers, patient-positioning controllers, and robotic surgical systems.

FIG. 4 further shows additional instruments that may be present during an example ACL repair. In particular, FIG. 4 shows an example guide wire or drill wire 424 and an aimer 426. The drill wire 424 may be used to create an initial or pilot tunnel through the bone. In some cases, the diameter of the drill wire may be about 2.4 millimeters (mm), but larger and smaller diameters for the drill wire 424 may be used. The example drill wire 424 is shown with magnified portions on each end, one to show the cutting elements on the distal end of the drill wire 242, and another magnified portion to show a connector for coupling to chuck of a drill. Once the surgeon drills the pilot tunnel, the surgeon and/or the surgical controller 418 (discussed more below) may then assess whether the pilot tunnel matches or closely matches the planned-tunnel path. If the pilot tunnel is deemed sufficient, then the drill wire 424 may be used as a guide for creating the full-diameter throughbore for the tunnel, and possibly also, in the case of the femoral portion of a procedure, for creating a counterbore associated with intercondylar notch to accommodate the graft. While in some cases the drill wire alone may be used when creating the pilot tunnel, in yet still other cases the surgeon may use the aimer 426 to help guide and place the drill wire 424 at the designed tunnel-entry location.

FIG. 4 also shows that the example system may comprise a calibration assembly 428. As explained in more detail in Quist et al., the calibration assembly 428 may be used to detect optical distortion in images received by the surgical controller 418 through the arthroscope 408 and attached camera head 410. Additional instruments will be present, such as a drill for drilling with the drill wire 424, various reamers for creating the throughbore and counterbore aspects of the tunnel, and various instruments for suturing and anchoring the graft in place. These additional instruments are not shown so as not to further complicate the figure.

FIG. 4 also shows that the example system may comprise an additional computing device with which a user, such as the surgeon, may interact. The computing device may be a tablet computer 614 in data communication with the surgical controller 418 and may provide various options for remotely reviewing and modifying a surgical plan, for controlling aspects of surgical controller 418, for reviewing, approving or rejecting segmentations and three-dimensional bone models, for assisting with registration of instruments and with registration of bone models to bones, and for generally aiding with other aspects of surgical guidance, as described herein.

The specification now turns to a workflow for an example ACL repair. The workflow may be conceptually divided into planning and repair. The repair workflow may be further conceptually divided into optical system calibration, model registration, tunnel-path planning, tunnel creation, and tunnel placement analysis. Each will be addressed in turn.

### PLANNING

In accordance with various examples, an ACL repair starts with imaging (e.g., X-ray imaging, computed tomography (CT), magnetic resonance imaging (MRI)) of the knee of the patient, including the relevant anatomy like the lower portion of the femur, the upper portion of the tibia, and the articular cartilage. The discussion that follows assumes MRI imaging, but again many different types of imaging may be used. The MRI imaging can be segmented from the image slices such that a volumetric model or three-dimensional model of the anatomy is created. Any suitable currently available, or after developed, segmentation technology may be used to create the three-dimensional model. More specifically to the example of ACL repair and specifically selecting a tunnel path through the femur, a three-dimensional bone model of the lower portion of the femur, including the femoral condyles, is created. Also, for specifically selecting a tunnel path through the tibia, a three-dimensional bone model of the upper portion of the tibia, including the tibial plateau, is created. Furthermore, the lateral and/or media menisci may be segmented from the image slices for use in creating respective three-dimensional models of the menisci.

Using the three-dimensional bone models, an operative plan is created. The creation of the operative plan may comprise choosing a planned-tunnel path through the femur, as described in Quist et al., including locations on the femur of the openings that will define the two ends of the femoral tunnel. The creation of the operative plan may also comprise choosing the location of a tunnel exit on the tibia - such as, for example, on the tibial plateau 115 - and optionally the location of a tunnel entry on the tibia and thus – with the location of the tunnel exit - an entire planned-tunnel path through the tibia. The exit openings of both the femoral and tibial tunnels may be selected by the surgeon to be the same as, or close to, the respective attachment locations of the native ACL to the femur and to the tibia.

The results of the planning may comprise: a three-dimensional bone model of the distal end of the femur; a three-dimensional bone model of a proximal end of the tibia; locations of an entry opening and an exit opening, on the femur, and thus a planned-tunnel path for the femur; and the locations of an entry opening and an exit opening, on the tibia, and thus a planned-tunnel path for the tibia. The results of the planning may also comprise: the relative positions of the femur and the tibia during imaging and, thus, the relative positions of the three-dimensional bone models of the femur and tibia with respect to each other. It will be appreciated that, because imaging is typically done while the joint is in extension, the relative positions of the femur and tibia that may be obtained from the images and determined during planning apply only insofar as the femur and tibia during the surgical procedure are in the same position as they were during imaging. That is, as the bones are part of a joint, a given orientation of the femur with respect to a given coordinate system is not fully determinative of the orientation of the tibia with respect to the same coordinate system. However, for the purpose of planning, the relative positions of the femur and tibia during imaging, and thus the relative positions of their three-dimensional models as a result of planning, may be useful at least for determining an estimate of the required length of a replacement graft as well as other surgical parameters. Such other surgical parameters may include tunnel throughbore diameters, tunnel counterbore diameters and depths, desired post-repair flexion, and the like. Furthermore, the relative positions of the femur and tibia, and other bones of the joint, during imaging may be useful for enabling three-dimensional bone models of each to represent their relative positions in order to aid with determining a risk of impingement by a bone on an inter-articular segment of a replacement ligament. It will be appreciated that, with respect to an ACL, the femur and tibia when the joint is in extension have the highest risk of impingement of the replacement ligament, whereas as the joint is moved through flexion the risk generally does not increase and may decrease. Furthermore, it will be appreciated that, with respect to an ACL, the when the joint is in flexion the replacement ligament will have more laxity whereas when the joint is in extension the replacement ligament will have more tension.

Where a meniscal root repair (MRR) may additionally be desirable, the results of planning may also comprise at least one additional planned-tunnel path in the tibia for a respective suture for binding. However, even where an MRR is planned, a surgeon may actually choose to initially define such an additional planned-tunnel path(s) for the MRR procedure only after finalizing the tibial tunnel path during the process of repair itself. That is, if during the repair the surgeon finds a particular planned-tunnel path for the ACL repair to be difficult to access for drilling or otherwise suboptimal for the procedure, the surgeon may modify the tunnel path. Modifying the tunnel path for the ACL repair may change the available options for the tunnel path for the MRR procedure.

With regard to the ACL repair, with the planned-tunnels through each of the femur and tibia with respect to the three-dimensional bone models of both the femur and the tibia having been established, and with the relative positions in space of the femur and tibia having been registered, an estimated length of replacement ligament may be calculated. A length of a replacement ligament may be calculated based on the length of the femoral planned-tunnel through which it is to be passed, the length of the tibial planned-tunnel through which it is to be passed, and the distance the replacement ligament is to traverse within the joint between the femur and the tibia. In particular, the distance the replacement ligament is to traverse through the joint will be dependent on the distance between the location of the tunnel exit at the tibia and the location of the tunnel exit at the femur, as calculated based on the relative positions of the three-dimensional models of the femur and tibia as determined during creation of the three-dimensional bone models.

It will be appreciated by the surgeon that the distance between the tunnel exits from the femur and the tibia will vary as the joint is bent from the full extension position in which they were held for imaging. It will also be appreciated by the surgeon that access to a joint for drilling tunnels may be provided only by bending the joint during repair. Because of this, it is probable that the distance between the tunnel exits during at least some portions of the repair itself will be different than the distance between the tunnel exits during imaging while in extension.

### REPAIR

The specification now turns to repair aspects. The repair aspects include steps and procedures for setting up the surgical system to perform the various repairs. It is noted, however, that some of the repair aspects (e.g., bone model/segmentation confirmation, optical system calibration, entry/exit point review and, if desired, adjustment), may take place before any ports or incisions are made through the patient’s skin, and in fact before the patient is wheeled into the surgical room. Nevertheless, such steps and procedures may be considered repair as they take place in the surgical setting and with the surgical equipment and instruments used to perform the actual repair.

The example ACL repair is conducted arthroscopically and is computer-assisted in the sense the surgical controller 418 is used for arthroscopic navigation within the surgical site. More particularly, in example systems the surgical controller 418 provides computer-assistance during the ligament repair by tracking location of various objects within the surgical site, such as the location of the particular bone (femur or tibia) being worked on within the three-dimensional coordinate space of the view of the arthroscope, and location of the various instruments (e.g., the drill wire 424, the aimer 426) within the three-dimensional coordinate space of the view of the arthroscope. The specification turns to brief description of such tracking techniques.

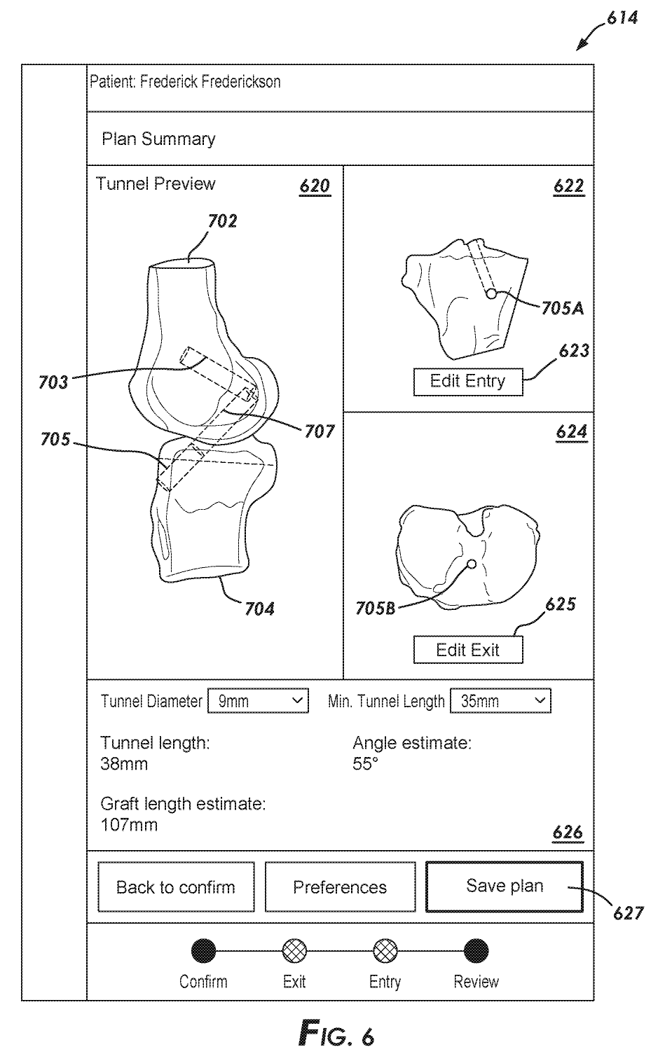

FIG. 5 shows a conceptual drawing of a surgical site with various objects within the surgical site. In particular, visible in FIG. 5 is a distal end of the arthroscope 408, a portion of a bone 500 (e.g., tibia), a bone fiducial 502 within the surgical site, a touch probe 504, and a probe fiducial 506. Each is addressed in turn.

The distal end of the arthroscope 408 is designed and constructed to illuminate the surgical site with visible light received by way of the light post 420. In the example of FIG. 5, the illumination is illustrated by arrows 508. The illumination provided to the surgical site is reflected by various objects and tissues within the surgical site, and the reflected light that returns to the distal end enters the arthroscope 408, propagates along an optical channel within the arthroscope 408, and is eventually incident upon a capture array within the camera head 410. The images detected by the capture array within the camera head 410 are sent electronically to the surgical controller 418 and displayed on the display device 414. In accordance with example systems, the arthroscope 408 has a single optical path through the arthroscope for capturing images of the surgical site, notwithstanding that the single optical path may be constructed of two or more optical members (e.g., glass rods, optical fibers). That is to say, in example systems and methods the computer-assisted navigation provided by the arthroscope 408, camera head 410, and surgical controller 418 is provided with the arthroscope 408 that is not a stereoscopic endoscope having two distinct optical paths separated by an interocular distance at the distal end endoscope.

During a surgical procedure, a surgeon selects an arthroscope with a viewing direction beneficial for the planned surgical procedure. Viewing direction refers to a line residing at the center of an angle subtended by the outside edges or peripheral edges of the view of an endoscope. The viewing direction for some arthroscopes is aligned with the longitudinal central axis of the arthroscope, and such arthroscopes are referred to as “zero degree” arthroscopes (e.g., the angle between the viewing direction and the longitudinal central axis of the arthroscope is zero degrees). The viewing direction of other arthroscopes forms a non-zero angle with the longitudinal central axis of the arthroscope. For example, for a 30^{o} arthroscope the viewing direction forms a 30^{o} angle to the longitudinal central axis of the arthroscope, the angle measured as an obtuse angle beyond the distal end of the arthroscope. In many cases for ACL repair, the surgeon selects a 30^{o} arthroscope or a 45^{o} arthroscope based on location the port created through the skin of the patient. In the example of FIG. 5, the view angle 510 of the arthroscope 408 forms a non-zero angle to the longitudinal central axis 512 of the arthroscope 408.

Still referring to FIG. 5, within the view of the arthroscope 408 is a portion of the bone 500, along with the bone fiducial 502, the touch probe 504, and the probe fiducial 506. The bone fiducial 502 is shown as a planar element having a pattern disposed thereon, though other shapes for the bone fiducial 502 may be used (e.g., a square block with a pattern on each face of the block). The bone fiducial 502 may be attached to the bone 500 in any suitable form (e.g., a fastener, such as a screw). The pattern of the bone fiducial is designed to provide information regarding the orientation of the bone fiducial 502 in the three-dimensional coordinate space of the view of the arthroscope 408. More particularly, the pattern is selected such that the orientation of the bone fiducial 502, and thus the orientation of the underlying bone 500, may be determined from images captured by the arthroscope 408 and attached camera head 410.

The probe fiducial 506 is shown as a planar element attached to the touch probe 504. The touch probe 504 may be used to “paint” the surface of the bone 500 as part of the registration of the bone 500 to the three-dimensional bone model, as described in Quist et al. Also, the touch probe 504 may also be used to indicate revised-tunnel entry locations in the case of changes to be made to the tunnel paths. The probe fiducial 506 is shown as a planar element having a pattern disposed thereon, though other shapes for the probe fiducial 506 may be used (e.g., a square block surrounding the touch probe 504 with a pattern on each face of the block). The pattern of the probe fiducial 506 is designed to provide information regarding the orientation of the probe fiducial 506 in the three-dimensional coordinate space of the view of the arthroscope 408. More particularly, the pattern is selected such that the orientation of the probe fiducial 506, and thus the location of the point of the touch probe 504, may be determined from images captured by the arthroscope 408 and attached camera head 410.

Other instruments within the view of the arthroscope 408 may also have fiducials, such as the drill wire 424 and aimer 426, but the additional instruments are not shown so as not unduly complicate the figure. Moreover, in addition to or in place of tracking location based on the view through the arthroscope 408, the location of the distal end of one or more of the instruments may be tracked by other methods and systems. For example, for devices that rigidly extend out of the surgical site (e.g., the aimer 426), the location may be tracked by an optical array coupled to the aimer and viewed through the camera 412, such as a stereoscopic camera. The location within the three-dimensional coordinate space of the camera 412 is then transformed into the three-dimensional coordinate space of the view of the example arthroscope to determine location of the distal end within the surgical site. A number of systems and techniques for registering an internal three-dimensional coordinate system such as that of an internal camera head 410 with an external three-dimensional coordinate system such as that of camera 412, are disclosed in PCT (International) Patent Application No. PCT/US2024/038340 filed on July 17, 2024, the contents of which are incorporated herein by reference.

The images captured by the arthroscope 408 and attached camera head 410 are subject to optical distortion in many forms. For example, the visual field between distal end of the arthroscope 408 and the bone 500 within the surgical site is filled with fluid, such as bodily fluids and saline used to distend the joint. Many arthroscopes have one or more lenses at the distal end that widen the field of view, and creating wider field of view causes a “fish eye” effect in the captured images. Further, the optical elements within the arthroscope (e.g., rod lenses) may have optical aberrations inherent to the manufacturing and/or assembly process. Further still, the camera head 410 may have various optical elements for focusing the images receives onto the capture array, and the various optical elements may have aberrations inherent to the manufacturing and/or assembly process.

In example systems, prior to use within each surgical procedure, the endoscopic optical system is calibrated to account for the various optical distortions. Various systems and methods for conducting a calibration are disclosed in Quist et al., and are not discussed further herein.

During planning, the surgeon may be provided with the opportunity to review and confirm three-dimensional models that have been generated based on image slices and segmentation processing on the image slices. For example, the surgeon may be provided with the image slices and the proposed segmentations of each, displayed on the display screen of tablet computer 614 or other similar computer, and may be enabled to use the computer to cycle through the image slices and corresponding segmentations to confirm or reject the segmentations. Various options may be made available via such a display screen during segmentation review, such as for confirming or rejecting a particular segmentation, for zooming in on a particular image slice to scrutinize features, for enabling the surgeon to review the segmentations in respect of just one bone at a time, and for accepting an overall segmentation of a bone as the basis for its three-dimensional models. Based on the acceptance by the surgeon of the segmentations for each bone, the three-dimensional bone models are created, or previously-generated three-dimensional bone models are modified and/or confirmed. In an example, the application executing on the surgical system for guiding the surgeon through a workflow may require the surgeon confirm the three-dimensional models in this way, prior to reviewing suggested tunnels and then registering the three-dimensional models to the bones of the patient for surgical guidance and repair.

Where a repair involves extending a replacement ligament between two bones, as in the case of ACL repair, aspects of tunnel path planning first conducted with respect to one of the bones imparts constraints on tunnel path planning with respect to the other bone. For example, when contemplating an ACL repair, a planned-tunnel path through a femur will correspond to a particular length of tunnel that will require a corresponding length of replacement ligament. That the planned-tunnel path will “take up” a corresponding portion of the length of the replacement ligament constrains how much there is left of the length of the replacement ligament to both traverse the region from one bone to the other (i.e., in the case of the ACL, the inter-articular region) and pass sufficiently into a tibial tunnel. Furthermore, a tunnel exit of a planned-tunnel path through a femur is considered by a surgeon when determining a tunnel exit of a planned-tunnel path through the tibia, with a view to recreating original knee kinematics, as well as when considering how much of the replacement ligament would be “taken up” by its traversal of the region between the bones.

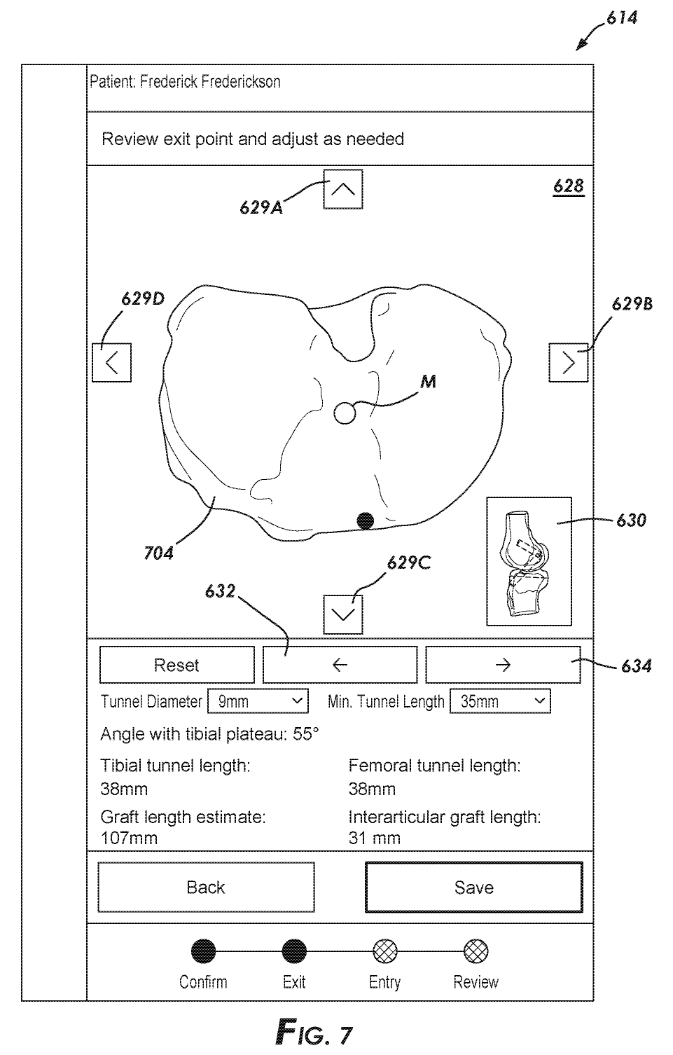

With the three-dimensional bone models having been confirmed, the surgeon may further be provided, at the time of repair, with the opportunity to review the bone models and the planned-tunnels and to make adjustments to tunnel entries, tunnel exits, and thus the planned-tunnel trajectories. FIG. 6 is a display screen of tablet computer 614 providing a plan summary of an ACL repair, according to an example. In a first panel 620 is displayed representations of the three-dimensional bone models of a portion of the femur 702 and a portion of the tibia 704, and planned-tunnels through each of these portions of the bones as well as an inter-articular path for a replacement ligament passing through the joint between these bones. These are shown in this example as femoral tunnel 703, tibial tunnel 705, and inter-articular path 707. In a second panel 622 is displayed an elevation view representation of just the portion of the tibia 704 and the planned-tunnel entry location 705A of the tibial tunnel 705. A user-interface button 623 is provided in the second panel 622 for enabling the surgeon to proceed to edit the planned-tunnel entry location 705A of the tibial tunnel 705. In a third panel 624 is displayed a plan view representation of just the portion of the tibia 704 and the planned-tunnel exit location 705B of the tibial tunnel 705. A user-interface button 625 is provided in the third panel 624 for enabling the surgeon to proceed to edit the planned-tunnel exit location 705B of the tibial tunnel 705. The surgeon is able to use the user interface, for example by touch-and-drag if the display screen is a touch display, to rotate the three-dimensional models in any of panels 620, 622 and 624, as desired. In a fourth panel 626 is displayed surgical parameters for each of the tunnels, including a dropdown enabling selection of the tunnel diameter, in this example set at 9mm, a dropdown enabling selection of the minimum tunnel length, in this example set at 35mm, and a display of other surgical parameters including tunnel length, in this example 38mm, a graft length estimate, in this example 107mm, and an angle estimate, in this example 55° (degrees). The surgeon is provided with a user-interface buttons to Save, and thereby confirm, the plan. If, however, the surgeon wishes to edit the planned-tunnel exit location 705B and/or the planned-tunnel entry location 705A, the surgeon may select the user-interface button in the second and/or third panels 622, 624 to enable display on tablet computer 614 of respective screens for editing. At the bottom of the display screen of tablet computer 614 is a stage bar indicating the stages of repair planning, including Confirm, Exit, Entry, and Review.

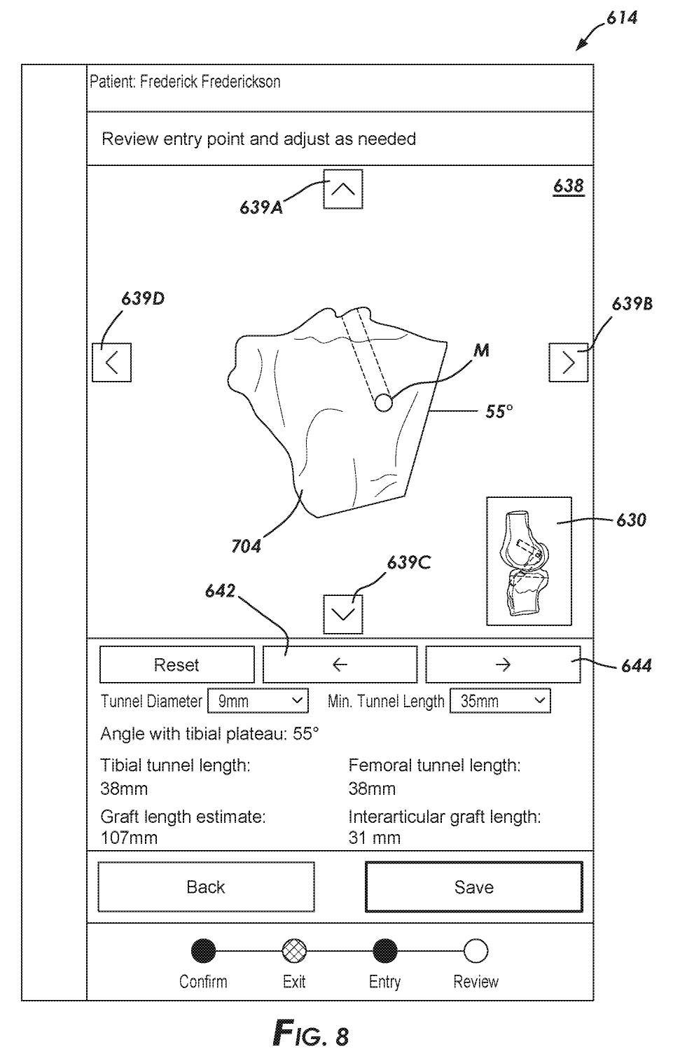

FIG. 7 is a display screen of tablet computer 614 providing an example edit screen displayed to the surgeon responsive to the selection of user-interface button 625 in the third panel 624 shown in FIG. 6, according to an example. The edit screen provides the surgeon with the opportunity to change the planned-tunnel exit location 705B of the tibial tunnel 705, in particular the location on the tibia 704 at which the tibial tunnel 705 is to open into the joint. In this example, the surgeon is provided with the plan view representation in a panel 628 of just the portion of the tibia 704, along with four positioning arrows 629A, 629B, 629C, 629D and a marker M at their center, the marker M initially being positioned at the planned-tunnel exit location 705B with respect to the tibia 704. Using any or all of the positioning arrows 629A-D, the surgeon is able to shift the plan view representation of the portion of the tibia 704 upwards, downwards, rightwards and/or leftwards such that the center marker M is repositioned with respect to the tibia 704. A small panel 630 provides a smaller view of the three-dimensional bone models and the planned-tunnel positions, similar to that provided in the first panel 620 of FIG. 6, to aid the surgeon with orientation as edits are being conducted. “Undo” and “Redo” arrow buttons 632, 634 are provided to enable the surgeon to revert back to a just-previous position of the marker M or to re-gain a position of the marker M pursuant to a reversion. Once the surgeon is satisfied with any edits that have been made to the planned-tunnel exit location 705B, the surgeon may select the Save button to return to the display shown in FIG. 6, but with the edits having been saved and stored for use in re-presentation of the bone models and tunnels in the display of FIG. 6. If the surgeon is satisfied with the plan as displayed in the panels of FIG. 6, the surgeon may select the Save Plan user interface button 627 to proceed to the repair itself.

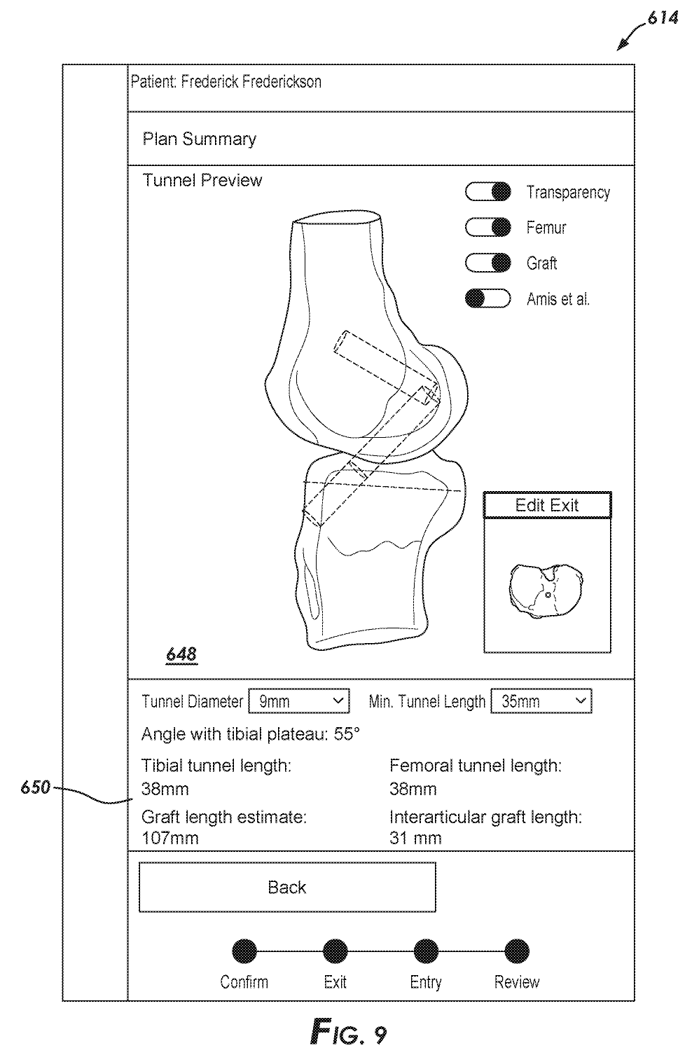

FIG. 8 is a display screen of tablet computer 614 providing an example edit screen displayed to the surgeon responsive to the selection of user-interface button 623 in the second panel 622 shown in FIG. 6, according to an example. The edit screen provides the surgeon with the opportunity to change the planned-tunnel entry location 705B of the tibial tunnel 705, in particular the location on the tibia 704 opposite that at which the tibial tunnel 705 is to open into the joint. In this example, the surgeon is provided with the plan view representation of just the portion of the tibia 705, along with four positioning arrows 639A, 639B, 639C, and 639D and a marker M at their center, the marker M initially being positioned at the planned-tunnel entry location 705A with respect to the tibia 704. Using any or all of the positioning arrows 639A-D, the surgeon is able to shift the plan view representation of the portion of the tibia 704 upwards, downwards, rightwards and/or leftwards such that the center marker M is repositioned with respect to the tibia 704. A small panel 630 provides the smaller view of the three-dimensional bone models and the planned-tunnel positions, similar to that provided in the first panel 620 of FIG. 6, to aid the surgeon with orientation as edits are being conducted. “Undo” and “Redo” arrow buttons 642, 644 are provided to enable the surgeon to revert back to a just-previous position of the marker M or to re-gain a position of the marker M pursuant to a reversion. Once the surgeon is satisfied with any edits that have been made to the planned-tunnel entry location 705A, the surgeon may select the Save button to return to the display shown in FIG. 6. If the surgeon is satisfied with the plan as displayed in the panels of FIG. 6, the surgeon may select the Save Plan user interface button 627 to proceed to the repair itself.

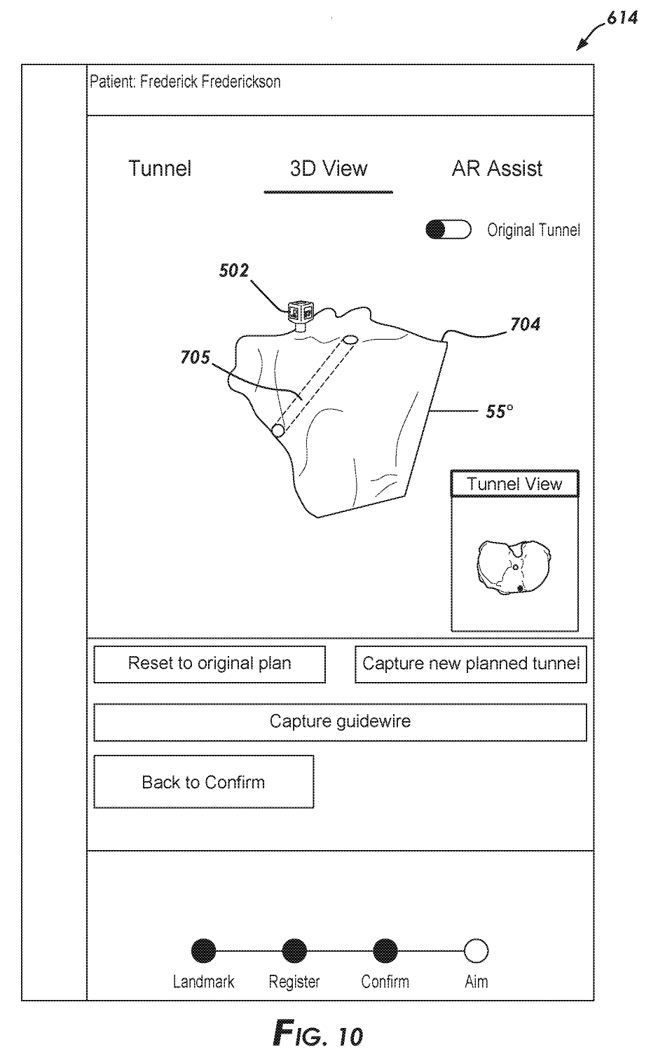

FIG. 9 is a display screen of tablet computer 614 displayed to the surgeon responsive to the selection of Save Plan in the display screen of FIG. 6. A first panel 648 displays representations of the three-dimensional bone models of a portion of the femur 702 and a portion of the tibia 704, and the saved planned-tunnels through each of these portions of the bones as well as the planned inter-articular pathway of the replacement ligament. The surgeon is provided with opportunities to go back and make further edits to the planned-tunnel exit location 705A and entry location 705B. A second panel 650 displays more granular surgical parameters, including the dropdowns for selecting tunnel diameter, in this example selecting 9mm, or selecting minimum tunnel length, in this example selecting 35mm, the angle with the tibial plateau, in this example 55 degrees, a tibial tunnel length, in this example 38mm, a femoral tunnel length, in this example 38 mm, the inter-articular graft length, in this example 31mm, and the estimated total graft length, in this example 107mm. Because the three-dimensional bone models are dimensionally accurate to the images that were processed to create them, such that planned-tunnel entry location 705A and planned-tunnel exit location 705B on the bone models selected by the surgeon can, once selected, be processed by the surgical controller to establish distances between the locations, and accordingly lengths for each of the planned-tunnels. It will also be appreciated that that the inter-articular graft length may be estimated based on the relative positions of the three-dimensional bone models based on the images that were processed to create them, and the planned-tunnel exit locations for each of the femoral tunnel 703 and the tibial tunnel 705 representing the ends of the inter-articular path 707. If the surgeon should make adjustments to the planned-tunnel exit locations (the locations at which the respective tunnels open into the joint), the inter-articular graft length will adjust accordingly, because the relative positions of the intercondylar notch and the tibial plateau, at least when in the position in which they were jointly imaged, are known. The estimated total graft length is the sum of the tibial tunnel length, the femoral tunnel length, and the inter-articular distance the replacement ligament will traverse, given the planned-tunnel exit locations into the joint. Informed by the estimated total graft length, the surgeon may thereafter prepare a replacement ligament, whether it be an autograft, an allograft, or an artificial ligament, to have this length and to have a thickness that can be accommodated by the selected tunnel diameter. If further guidance is desired, a surgeon may be provided with the opportunity, via a slider user interface element, to display an Amis et al. grid in association with a representation of the tibia thereby to help confirm, or to edit, placement the tibial tunnel 705 relative to that which is accepted in the literature. In the display of FIG. 9, the surgeon may also adjust what is displayed, including whether to show the femur 702 at all, whether to show the graft, and a transparency option.

Data respecting this tunnel path planning done in respect of one of the bones is stored by the surgical controller 418 and made available for guiding tunnel path planning being done in respect of the other bone. It may be preferable for such planning to be done first with respect to the femur, such that data respecting the femoral tunnel 703 is available to the surgical controller while the surgeon is planning the tibial tunnel 705. For example, a femoral software application executed by the surgical controller 418 may provide workflow and guidance to a surgeon for planning and forming a tunnel through a femur, and may capture data defining the femoral planned-tunnel – a first of two tunnels to be made for the ACL repair - that results from such planning, such as the position and orientation of the femoral planned-tunnel 703 with respect to the three-dimensional model of the femur, the length of the femoral planned-tunnel, the tunnel exit, the angle of entry and exit of the planned-tunnel with respect to the femur, and other data calculated by the surgical controller 418. Such captured data may be stored in a data file or other data structure that is stored by and made available to the surgical controller 418 for when the surgeon subsequently begins using a tibial software application for planning, confirming, pre-operative planning, and forming a corresponding tibial tunnel 705 through the tibia. The data file or other data structure may be stored after planning, using the femoral software application, is complete, and may be automatically retrieved or selected by a surgeon, and made available to the tibial software application executing on the surgical controller 418. In this way, as the surgeon is making plans for a tibial planned-tunnel using the tibial software application, and as the surgeon is within the joint conducting the repair by orienting tracked surgical instruments and determining whether certain tunnel trajectories can actually be achieved, as will be described, the data about the femoral planned-tunnel 703, as well as the overall replacement ligament length and other dimensions such as replacement ligament thickness, can be used by the surgical controller 418 to provide the surgeon with various kinds of guidance so that planning and drilling of the tibial tunnel 705 can be conducted with the benefit of the constraints on tunnel position, replacement ligament dimensions, and other such constraints that have been imparted by the planning of the femoral tunnel 703 that had already taken place.

The next example step in the repair procedure is the registration of the bone model(s). During the repair, the three-dimensional bone models are provided to the surgical controller 418, along with data about their relative poses at the time they were being imaged, typically for example as described herein while they were in extension. Again using the example of ACL repair, and specifically computer-assisted navigation for tunnel paths through the femur, the three-dimensional bone model of the lower portion of the femur is provided to the surgical controller 418. Thus, the surgical controller 418 receives the three-dimensional bone model, and assuming the arthroscope 408 is inserted into the knee by way of a port through the patient’s skin, the surgical controller 418 also receives video images of the femur. In order to relate the three-dimensional bone model to the images received by way of the arthroscope 408 and camera head 410, the surgical controller 418 registers the three-dimensional bone model to the images of the femur received by way of the arthroscope 408 and camera head 410.

In accordance with example methods, a fiducial marker or bone fiducial (e.g., bone fiducial 502 of FIG. 5) is attached to the femur. The bone fiducial placement is such that the bone fiducial is within the field of view of the arthroscope 408, but in a location spaced apart from the expected tunnel entry/exit point through the lateral condyle. More particularly, in example cases the bone fiducial 502 is placed within the intercondylar notch superior to or above the expected location of the femoral tunnel 703 through the lateral condyle.

Disclosed in Quist et al. are systems and techniques for, for the purpose of a femoral portion of the ACL repair, registering the three-dimensional bone model of the femur to images of the femur received by way of the arthroscope 408 and camera head 410.

Similarly, for the purpose of a tibial portion of the ACL repair, in order to register the three-dimensional bone model of the tibia to images of the tibia received by way of the arthroscope 408 and camera head 410, similar systems and techniques to those disclosed in Quist et al. may be used. More particularly, using the example of computer-assisted navigation for tunnel paths through the tibia, the three-dimensional bone model of the upper portion of the tibia is also provided to the surgical controller 418. Thus, the surgical controller 418 receives the three-dimensional bone model and, assuming the arthroscope 408 is inserted into the knee by way of a port through the patient’s skin, the surgical controller 418 also receives video images of the tibia. In order to relate the three-dimensional bone model to the images received by way of the arthroscope 408 and camera head 410, the surgical controller 418 registers the three-dimensional bone model to the images of the tibia received by way of the arthroscope 408 and camera head 410.

In accordance with example methods, a fiducial marker or bone fiducial (e.g., bone fiducial 502 of FIG. 5, which may be a different actual element than that attached to the tibia, and may have different fiducial markings) is attached to the tibia. The bone fiducial placement is such that the bone fiducial is within the field of view of the arthroscope 408, but in a location spaced apart from the expected tunnel exit aperture through the tibial plateau. In examples, the bone fiducial 502 for the tibial portion of the ACL repair may be placed on the tibial plateau.

As explained in Quist et al., for the femoral portion of an ACL repair, a video display may show portions of a femur and an associated bone fiducial. The display may be shown, for example, on the display device 414 associated with the device cart 402, or any other suitable location. Similarly, for the tibial portion of an ACL repair, the video display may show portions of a tibia and an associated bone fiducial in the tibia.

While various kinds of bone fiducial, each serving as a machine-readable marker, may be used, in an example the bone fiducial may comprise a cube member. Of the six outer faces of the cube member, the bottom face may be associated with an attachment feature (e.g., a screw). The bottom face will be close to, or will abut, the bone when the bone fiducial is secured in place, and thus will not be visible in the view of the arthroscope 408. The outer face opposite the bottom face may include a placement feature used to hold the bone fiducial prior to placement, and to attach the bone fiducial to the underlying bone. Of the remaining four outer faces of the cube member, each of the four outer faces has a machine-readable pattern thereon, and in some cases each machine-readable pattern is unique. Once placed, the bone fiducial represents a fixed location on the outer surface of the bone in the view of the arthroscope 408, even as the position of the arthroscope 408 is moved and changed relative to the bone fiducial. Initially, the location of the bone fiducial with respect to the three-dimensional bone model is not known to the surgical controller 418, hence the need for the registration of the three-dimensional bone model.

In or order to relate or register the bone visible in the video images to the three-dimensional bone model, the surgical controller 418 is provided and thus receives a plurality of locations of an outer surface of the bone. For example, the surgeon may touch a plurality of locations using the touch probe 504. As discussed in Quist et al., the touch probe 504 comprises a probe fiducial 506 visible in the video images captured by the arthroscope 408 and camera head 410. The physical relationship between the distal end of the touch probe 504 and the probe fiducial 506 is known by the surgical controller 418, and thus as the surgeon touches each of the plurality of locations on the outer surface of the bone, the surgical controller 418 gains an additional “known” locations of the outer surface of the bone relative to the bone fiducial 1006. Given that the touch probe 504 is a relatively inflexible instrument, in other examples the tracking of the touch probe 504 may be by optical tracking of an optically-reflective array outside the surgical site (e.g., tracking by the camera 412) yet attached to the portion of the touch probe 504 inside the surgical site.

In some cases, particularly when portions of the outer surface of the bone are exposed to view, receiving the plurality of locations of the outer surface of the bone may involve the surgeon “painting” the outer surface of the bone. “Painting” is a term of art that does not involve application of color or pigment, but instead implies motion of the touch probe 504 when the distal end of the touch probe 504 is touching bone.

Further details of a registration procedure for a three-dimensional bone model of a femur for use during the femoral portion of the repair are described and depicted in Quist et al., and will not be described further herein. However, it is the case that the same or a similar registration procedure as disclosed in Quist et al. may be conducted for a three-dimensional bone model of a tibia, for use during the tibial portion of the ACL repair. Once the surgeon approves the registration, tunnel path planning may follow.

### TUNNEL PATH PLANNING

Using the three-dimensional bone model an operative plan is created that comprises a planned-tunnel path through the bone, including locations of the apertures into the bone that define the ends of the tunnel. In some cases, however, when contemplating the repair – when actually orienting instruments such as aimers and drill wires - the surgeon may elect not to use a planned-tunnel path, and thus elect not use the planned entry location, exit location, or both. Such an election can be based any of a number of reasons. For example, intraoperatively the surgeon may not be able to access the location of a tunnel exit for the planned-tunnel path with an instrument, and thus may need to move the location of the exit to ensure sufficient access by the instrument. As another example, during the repair itself the surgeon may determine that the location of the tunnel exit is misaligned with the attachment location of the native ACL to the femur or tibia. Further still, during the intraoperative procedure the surgeon may determine the location of the tunnel exit is too close to the posterior wall of the femur, or that the angle of approach is too small, increasing the likelihood of a bone chip sometimes referred to as a “back wall blowout,” or – for the tibial portion of the repair - too close to the edge of the tibial plateau. Regardless of the reason for the surgeon’s election to change the tunnel path, even if the surgeon has made adjustments, prior to repair, to the tunnel trajectories, in example systems the surgical controller 418 enables the surgeon to intraoperatively select one or more of a revised-tunnel entry and a revised-tunnel exit, and thus select a revised-tunnel path through the bone.

Quist et al. describes and depicts the display of video showing intraoperative changes to the tunnel path, in accordance with at least some embodiments. Such a display may be shown, for example, on the display device 414 (FIG. 4) associated with the device cart 402 (FIG. 4), or any other suitable location. However, for any number of reasons, the surgeon may elect to modify the tunnel entry location and/or the tunnel exit location, and thus modify the planned-tunnel path. Thus, the surgeon may provide to the surgical controller 418 (FIG. 4), and thus the surgical controller 418 may receive, a revised-tunnel entry location or revised-tunnel exit location. Providing the revised-tunnel entry may comprise the surgeon touching the proposed location on the bone shown in the video images with a tracked instrument, such as the touch probe 504 (FIG. 5) or the aimer 426 (FIG. 4). In some cases, the surgeon may select the revised-tunnel entry based solely on what the surgeon sees of the bone shown in the video images. As a more specific example, the surgeon may select and provide the revised-tunnel entry based on a location that can be reached by the aimer 426. In yet still other cases, for the femoral portion of the repair, the surgical controller 418 may generate a simulated fluoroscopic images from the three-dimensional bone model, and project thereon a Bernard & Hertel Quadrant or grid. The surgeon may then select the revised-tunnel entry with the additional guidance provided by the Bernard & Hertel Quadrant. For the tibial portion of the repair, the surgical controller 418 may generate such images and an Amis et al. grid for guidance.

In many cases, creating a revised-tunnel path involves selecting the revised-tunnel entry, and the other features of the tunnel remain unchanged, such as the tunnel exit location. However, in some cases, the surgeon may change both the tunnel entry location and the tunnel exit location. Thus, in yet still further examples, the surgeon may provide the surgical controller 418, and thus the surgical controller 418 may receive, a revised-tunnel exit location or just revised-tunnel exit 1308. Providing the revised-tunnel exit may comprise the surgeon touching the proposed location on the bone shown in the video images with a tracked instrument, such as the touch probe 504. In various examples, with the revised-tunnel entry and optionally the revised-tunnel exit, the surgical controller calculates a revised-tunnel path through the bone of the patient, and displays the revised-tunnel path on the display device. As described in Quist et al., the surgical controller 418 may provide information to the surgeon regarding the relationship between the planned-tunnel path and the revised-tunnel path. In particular, the example video display may further include various parameters to help the surgeon assess the viability of the newly created revised-tunnel path. For example, the surgical controller 418 may calculate and provide a value indicative of overlap of the planned-tunnel path and the revised-tunnel path, and the surgical controller 418 may display a visual representation of the value indicative of overlap. The visual representation of the value indicative of overlap may be a numerical value shown as a percentage. The overlap as a percentage conceptually may span from 0% to slightly less than 100%, as at 100% overlap the revised-tunnel path and the planned-tunnel path would be the same. In some cases, the surgical controller 418 may calculate the value indicative of overlap as a percentage taking into account the expected tunnel diameters. If any portion of the planned-tunnel path intersects any portion of the revised-tunnel path, then that intersection is considered overlap. In other cases, the overlap may be calculated with respect to a planned pilot tunnel and a revised pilot tunnel. The planned-tunnel path and revised-tunnel path may be selected to have sufficient separation to be visible and distinguishable in the view of the display; however, in practice the locational change as between the planned-tunnel path and the revised-tunnel path may be slight, and thus have significant overlap taking into account the expected diameter of the tunnel path.

In still further examples the surgical controller 418 (FIG. 4) may calculate and provide an entry-location offset as between the planned-tunnel entry and the revised-tunnel entry, and the surgical controller 418 may display a visual representation of the offset. The visual representation of the offset may be a numerical value shown in a measurement unit (e.g., millimeters). The surgical controller 418 may also calculate and provide an exit-location offset between the planned-tunnel exit and the revised-tunnel exit, and the surgical controller 418 may display a visual representation of the offset in the measurement unit. Again, the example planned-tunnel path and revised-tunnel path may be selected to have sufficient separation to be visible and distinguishable in the display; however, in practice the locational change as between the planned-tunnel path and the revised-tunnel path may be slight, and thus have smaller offsets. In many cases the revised-tunnel exit will be identical to the planned-tunnel exit, and in such cases the exit offset will be zero.

Still considering information provided to the surgeon regarding the planned-tunnel path and the revised-tunnel path, in yet still further examples the surgical controller 418 (FIG. 4) may calculate and provide a value indicative of back wall blowout. The value indicative of back wall blowout may have two example aspects – a quantized blowout potential (e.g., low, medium, and high), and a numerical value indicative of back wall blowout potential. In example cases, the numerical value indicative of back wall blowout potential may be a distance, calculated by the surgical controller 418, as between the expected outside diameter of the revised-tunnel path and the outside surface of the bone of the three-dimensional bone model. More particularly still, in example cases the numerical value indicative of back wall blowout is the calculated shortest distance between the expected inside diameter of the revised-tunnel path and the outside surface of the three-dimensional bone model. In some cases, quantized blowout potential is related to the numerical value indicative of back wall blowout potential, for example: “low” blowout potential may be displayed when the shortest distance between the expected inside diameter of the revised-tunnel path and the outside surface of the three-dimensional bone model is 8 mm or more; “medium” blowout potential may be displayed when the shortest distance between the expected inside diameter of the revised-tunnel path and the outside surface of the three-dimensional bone model is between 4 and 8 mm; and “high” blowout potential may be displayed when the shortest distance between the inside diameter of the revised-tunnel path and the outside surface of the three-dimensional bone model is 4 mm or less. In many cases the tunnel path through a femur will have a counterbore associated with the intercondylar notch side of the tunnel. Similarly, in many cases the tunnel path through a tibia will have a counterbore associated with the tibial plateau side of the tunnel. It follows that the counterbore portion of the tunnel may have a greater inside diameter than that of the portion of the tunnel opposite the intercondylar notch/tibial plateau, and in an example case the surgical controller 418 considers the expected inside diameter of the counterbore when calculating the values indicative of back wall blowout potential.

Regardless of the precise information provided to the surgeon regarding the relationship between the planned-tunnel path and the revised-tunnel path, if the surgeon so elects based on the provided information, the revised-tunnel path may be scrapped and selecting a revised-tunnel entry may begin anew. The specification continues with the assumption that the surgeon selected a revised-tunnel path for use; however, it is not necessary that a revised-tunnel path be selected in every case.

Where planning for the tibial tunnel is concerned, the surgeon may be provided with guidance established by positional and ligament length constraints that were established when planning the femoral tunnel, and when harvesting or otherwise specifying the replacement ligament and preparing it for use. In the case of the tibial tunnel, the surgeon will generally have increased flexibility in tunnel placement where, for outside-in aiming, the location at which the tunnel exits into the intra-articular region is more important than the location at which the tunnel enters at the anterior of the tibia. This is due to it being generally desirable that, if possible, the tunnel exit location is within the original ACL footprint. The tip of an aimer can be placed by the surgeon at the location of the tunnel exit with the aid of an overlay – a digital marker – being displayed at, and tracking, the planned exit location as it appears in the video being captured by the arthroscopic camera of the surgical site including the bone marker. Furthermore, while the bone fiducial and a fiducial of the aimer is within the field of view of the camera head 408, the aimer can be pivoted about the location of the exit while maintaining the tip of the aimer at that location, by a surgeon wishing to bring the overall aimer to an appropriate position for drilling the tibial tunnel. The surgeon can be guided in positioning of the aimer by the presentation of a visual overlay on a display device in association with the presentation of the arthroscopic video, for example an overlay graphic of the candidate tunnel as it would appear based on a drilling axis being tracked based on tracking the current position and orientation (the “pose”) of the aimer, the current aimer angle, and a diameter of the tunnel selected or specified by the surgeon that could be drilled along that drilling axis. Additionally or in the alternative, the surgeon can be guided in positioning of the aimer by the presentation of a visual representation of a three-dimensional model of the aimer itself and/or of its longitudinal drilling axis and/or of a tunnel that would result were drilling to be done along the longitudinal drilling axis, in association with presentation of the three-dimensional model of the bone. The aimer angle may be received as a result of the surgical controller 418 processing video frames captured of the aimer itself in order to determine, based on its configuration or information displayed on the aimer itself, its aimer angle. Such a display in association with the three-dimensional model may be presented on the same display as the arthroscopic video, or on a different display, such as a tablet computer.

As different aimers have different geometries, the surgical controller 418 may be provided, and thus the surgical controller 418 may receive, information representative of an identification of the aimer being manipulated during this planning. This identification of the adjustable aimer may be specified by the surgeon by selection from a dropdown user interface element that lists kinds of aimers or that lists individual aimers. Alternatively or in some combination, the fiducial markings on the aimer being manipulated may encode the kind of aimer being manipulated or the individual aimer itself. In this way, the surgical controller 418 may display guidance overlays and other data based on the predetermined geometry of the aimer actually being manipulated. Such individual or kinds of aimers, or the fiducial markers to be associated therewith, may have been calibrated with the use of systems or methods described in United States Patent Application Serial No. 18/777,812 to Dos Santos Raposo et al. filed on July 19, 2024, the contents of which are incorporated herein by reference. The user may select the predetermined geometry of the adjustable aimer and provide this user selection to the surgical controller 418. In an example, an adjustable aimer may be configured to provide a fixed number of discrete bullet pathways from which the surgeon can choose, rather than providing the surgeon with the freedom to set any pathway between a maximum and minimum angle. In such a case, the surgical controller 418 may present a user interface element on, for example, the tablet computer 614, that includes a representation of such an adjustable aimer and provides the surgeon with the option of selecting which one of the fixed number of discrete bullet pathways that the surgeon has chosen for the adjustable aimer itself. In this way, the surgical controller 418 is provided with the aimer angle.

Furthermore, in some cases the surgeon may plan to drill an additional tunnel or tunnels in a bone for another repair, such as for a meniscal root repair. Where there are to be multiple tunnels through a bone, it may be useful to provide guidance to the surgeon as to the relative planned positions of the tunnels within the bone, as well as guidance during revisions of one or more of their paths so that any risk of multiple tunnels converging can be understood by the surgeon and planned for.

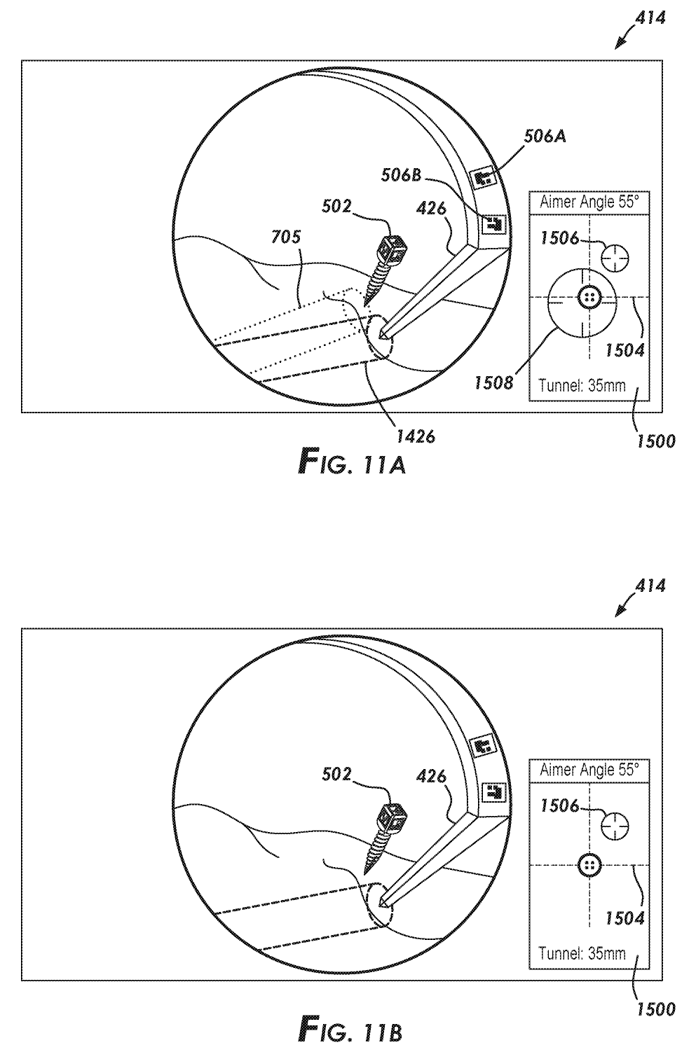

FIG. 10 is a display screen of a tablet computer 614 showing the three-dimensional model of the tibia 704 presented to the surgeon pursuant to placing a bone fiducial 502 at a fixed location on the tibia and pursuant to the three-dimensional bone model of the tibia having been registered to the bone in the manner that has been described in Quist et al. with respect to the femur. The bone fiducial 502 shown in FIG. 10 is a representation of the actual bone fiducial 502 that is affixed to the actual tibia within the surgical site. The planned-tunnel 705 through the tibia 704 is displayed in FIG. 10, positioned with respect to the representation of the bone fiducial 502, in order to aid the surgeon with orienting him or herself with respect to what is captured within the field of view of the arthroscope 408 and attached camera head 410 (FIG. 4).

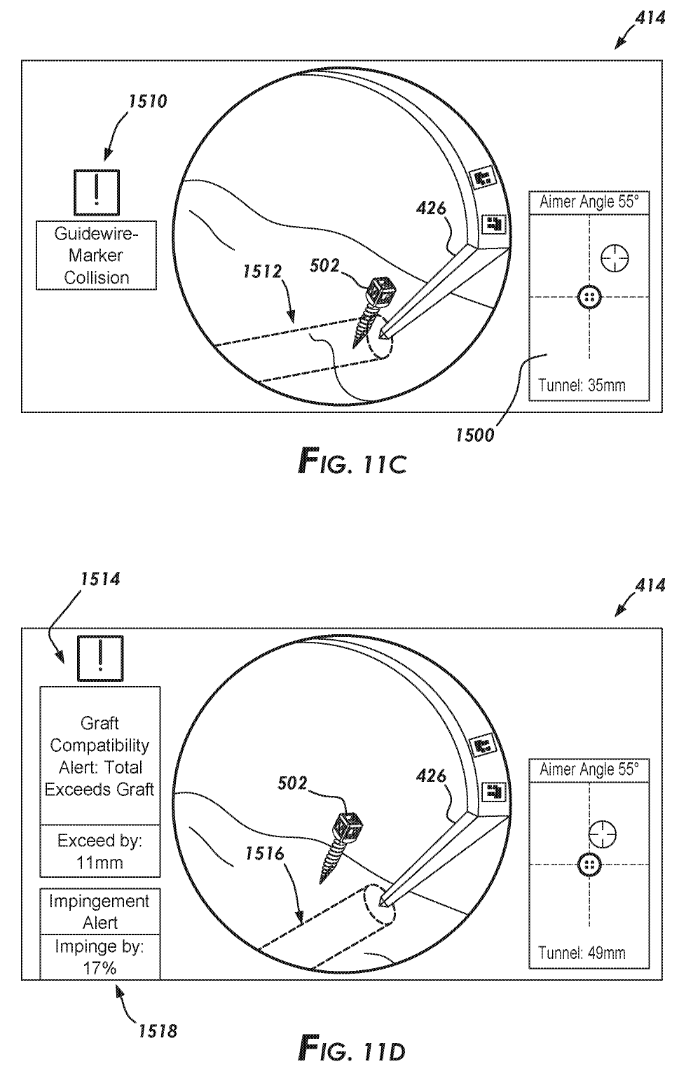

FIG. 11A is an example video display showing computer guidance for placement of a pilot ACL tunnel. The display may be shown, for example, on the display device 414 (FIG. 4) associated with the device cart 402 (FIG. 4), or any other suitable location.

In particular, FIG. 11A shows, in the main portion of the display, a portion of the bone in the video images as captured by the arthroscope 408 (FIG. 4), a depiction the revised-tunnel path 705, and the proximal end of the example aimer 426 for an outside-in pilot hole. In the lower right corner, FIG. 11A shows an example graphic 1500 that shows the relative locations of the longitudinal central axis of the aimer 426 (which longitudinal central axis corresponds to the drill axis for the drill wire 424 (FIG. 4)), indicated at 1426, and the longitudinal central axis of the revised-tunnel path 705.

Referring initially to the aimer 426, the portion of the aimer 426 visible in the view captured by the arthroscope 408 (FIG. 4) is its tip, and the aimer includes a tube having a throughbore for aligning a drill axis of a drill wire with which the aimer 426 is to be used, with the tip. In this example, the alignment of the throughbore with the tip corresponds to the drill axis of the drill wire. It will be appreciated that, in other examples, an aimer may have a geometry with a tip that is not precisely coincident with its drill axis (i.e. a notional line coincident with the drill axis would not strictly pass through the end of the tip), by virtue of the throughbore or other structure defining the path of a drill wire through the aimer not itself being necessarily perfectly aligned with the tip of that aimer, or even designed to be perfectly aligned with the tip of the aimer. However, for the purpose of this particular example, an aimer will be described in which the alignment of the throughbore with the tip of aimer 426 does correspond to the drill axis of the drill wire. In example cases the surgeon uses the aimer 426 to hold and guide the drill wire 424 (FIG. 4). The aimer 426 is selected to have an inside diameter to create a slip fit with the drill wire such that longitudinal central axis of aimer 426 is coaxial with the longitudinal central axis of the drill wire 424. Moreover, the portion of the aimer 426 visible in the video images captured by the arthroscope includes an aimer fiducial 506. Based on the video images, the surgical controller 418 may “see” the machine-readable aimer fiducial 506, indicated in this example with two machine-readable fiducial markers 506A, 506B, and thus calculate both the location of the proximal end of the aimer 426 and the orientation of the longitudinal central axis of the aimer 426 and accordingly the drill wire 424, with the locations and orientations thus known in the three-dimensional coordinate space of the view captured by the arthroscope 408. In this manner, by processing video frames captured by the arthroscope, and processing both the bone fiducial 502 and the fiducial markers 506A, 506B of aimer 426, the surgical controller 418 can track the pose (location and orientation) of the aimer 426 with respect to the bone fiducial 502 and, accordingly, with respect to three-dimensional model of the tibia. This, in turn, due to the three-dimensional model of the tibia having been registered to the tibia, permits tracking of the aimer 426 with respect to the tibia itself. It will be appreciated that, where the aimer is adjustable, data concerning the current angle of an adjustable aimer 426 may be provided to the surgical controller 418 to aid with tracking the drill axis, either manually by the surgeon or, depending on the construction of the aimer itself, automatically.

During the initial placement of the aimer 426, the surgeon may rely upon viewing the relative locations of the aimer 426 and the revised-tunnel path 705 in the video images. However, for fine alignment of the aimer 426 with the revised-tunnel entry 705A, and alignment of the longitudinal central axis of the aimer 426 with the longitudinal central axis of the revised-tunnel path 705, in example cases surgeon may rely upon the graphic 1500 generated and shown by the surgical controller 418 (FIG. 4). In particular, in accordance with example systems the surgical controller 418, receiving the video images capture by the arthroscope 408 (FIG. 4) and camera head 410 (FIG. 4), tracks location of the proximal end of the aimer 426 relative to the revised-tunnel entry location 705A, and displays the graphic 1500 on the display device that shows the relative locations of the revised-tunnel entry location 705A and both the distal and the proximal ends of the aimer 426.

In particular, in the lower right corner of the example display is the graphic 1500 including a tunnel-path target 1504 representing the revised-tunnel entry location 705A and illustratively shown as an extended length crosshair. Further in the example graphic 1500 is a distal-end target 1506 representing the position of the distal end of the aimer 426 and illustratively shown as a crosshair embedded within a smaller circle. In example systems and methods, the surgical controller 418 displays the tunnel-path target 1504 as fixed in place on the display device, and further displays the distal-end target 1506 at a variable location to depict the relative positioning of the distal end of the aimer 426 and the revised-tunnel entry location 705A. The example video display of FIG. 11A may be shown on a display device having a size (e.g., measured diagonally) of 120 centimeters or more, while the relative spacing between the distal end of the aimer 426 and the revised-tunnel entry location 705A may be just a few centimeters. Thus, the relative locations shown by the tunnel-path target 1504 and the distal-end target 1506 may include a scale factor to provide scaled visual feedback to the surgeon. The goal of the surgeon is to place the distal-end target 1506 aligned with the tunnel-path target 1504 before beginning drilling of the pilot tunnel using the drill wire 424 (FIG. 4). The drill wire 424 may be disposed within the aimer 426 during the alignment process, or the surgeon may align the aimer 426 prior to telescoping the drill wire 424 into the aimer 426.

There are at least two alignments for the surgeon to consider when placing the aimer 426 for drilling of the pilot tunnel: 1) having the actual tunnel entry location close to or aligned with the revised-tunnel entry location 705A; and 2) having the longitudinal central axis of the pilot tunnel close to or coaxial with the longitudinal central axis of the revised-tunnel path 705. Placing the distal-end target 1506 closely aligned with the tunnel-path target 1504 only addresses the first alignment consideration. The distal-end target 1506 may be precisely aligned with the tunnel-path target 1504, yet if the pilot tunnel was drilled the tunnel direction could differ substantially from the revised-tunnel path 705. In order to enable better axial alignment in accordance with further examples, the surgical controller 418, still receiving the video images capture by the arthroscope 408 (FIG. 4) and camera head 410 (FIG. 4), tracks the orientation of the longitudinal central axis of the aimer 426 relative to the longitudinal central axis of the revised-tunnel path 705, and displays a graphic on the display device that shows the relative orientations of the central axes.

Again referring to the graphic 1500 in the lower right corner, in example systems and methods the surgical controller 418 (FIG. 4) further generates and displays a proximal-end target 1508 representative of a proximal portion of the aimer 426 and illustratively shown as a partial crosshair embedded within a larger circle. In example systems and methods, the surgical controller 418 displays the proximal-end target 1508 at a variable location relative to distal-end target 1506 to show the orientation of the longitudinal central axis of the aimer 426 relative to the longitudinal central axis of the revised-tunnel path 705. That is, in the graphic 1500 the longitudinal central axis of the revised-tunnel path 705 may be considered to be perpendicular to the front face of the display device 414 (FIG. 4) and disposed at the intersection or center of the tunnel-path target 1504. The longitudinal central axis of the aimer 426 may be considered to be a line extending between the centers of the proximal-end target 1508 and the distal-end target 1506. The goal of the surgeon is to align the proximal-end target 1508 with the distal-end target 1506, and to have the aligned crosshairs 1508/1506 aligned with the tunnel-path target 1504. When all the crosshairs are aligned, the longitudinal central axis of the aimer 426, the drill wire 424 (FIG. 4) within the aimer 426, and the revised-tunnel path 705 should be coaxial.

It will be appreciated that, where the tibial tunnel 705 in particular is concerned, the exit location 705B of the revised-tunnel path 705 is more important than the entry location 705A. It may therefore be useful to provide the surgeon with more flexibility of tunnel placement by not constraining the entry location 705A to be positioned as established during planning. In accordance with an example, the surgeon may select to be provided with guidance that contemplates adhering to the whole revised-tunnel path trajectory, or simply guidance that contemplates adhering only to the revised exit location 705B such that an outside-in tunnel may be drilled starting from a different location than entry location 705A, but that ultimately exits at, or suitably close to, exit location 705B.

FIG. 11B is an example video display showing computer guidance for placement of a pilot tunnel, where the surgeon has selected exit-only navigation. FIG. 11B is very similar in presentation to FIG. 11A, but differs in that proximal-end target 1508 is not presented in graphic 1500. In this manner, while the drill axis of the aimer 426 is still tracked in order to provide guidance to the surgeon should constraints such as exit location, length of the tibial tunnel, angle with the tibial plateau, and blowout thresholds be violated, with exit-only navigation it is not held up by surgical controller 418 as a constraint, per se, that the drill axis must align with the revised-tunnel entry point.

Regarding constraints during guidance, FIG. 11C is an example video display showing computer guidance for placement of a pilot tunnel, where the surgeon has selected exit-only navigation. In the main portion of the display, the aimer 426 and field of view of the arthroscope have shifted such that the longitudinal central axis of the aimer 426 has shifted. In the particular condition shown in FIG. 11C, the longitudinal central axis of the aimer 426 coincides with the threads of bone fiducial 502. That this is the case is determined automatically by the surgical controller 418 by processing the video images to track the longitudinal central axis of the aimer 426 with respect to the three-dimensional model, and thus with respect to the tibia itself. During registration of the three-dimensional bone model, the surgical controller 418 has also registered the pose of the particular bone fiducial 502, including its envelope (i.e., the extent and shape of the three-dimensional space it occupies, in the frame of reference of the bone fiducial 502) with respect to the three-dimensional model, and thus with respect to the tibia itself. By tracking the longitudinal central axis of the aimer 426, and with consideration of at least a surgical parameter representing the diameter of the pilot tunnel, the surgical controller 418 calculates an envelope of the pilot tunnel with respect to the frame of reference of the bone fiducial 502 based on the current pose of the aimer 426. It will be appreciated that a buffer zone may be included in the envelope of the pilot tunnel which corresponds to a larger diameter envelope than simply that which would be required of an actual pilot tunnel. By processing both the envelope of the bone fiducial 502 and the envelope of the pilot tunnel, the surgical controller 418 is able to calculate whether they intersect, or “collide”, at any point. If the surgical controller 418 calculates that the envelopes collide, this represents a risk of a drill wire colliding with the bone fiducial 502 should a pilot tunnel be drilled according to the current pose of the aimer 426.

In this example, a first alert 1510 for alerting the surgeon as to the risk of collision - a text alert - is shown prominently in a position on display device 414 that is adjacent the main display. Also, in this example, a second alert 1512 for alerting the surgeon as to the risk of collision – a graphical alert – is shown in the form of the representation of the longitudinal central axis of the aimer 426 changing in visual appearance. In this way, the surgeon when navigating within the surgical site can be provided with visual feedback as to the appropriateness of a particular position of aimer 426. In this example, the visual appearance change is a change in the line pattern of the representation of the longitudinal central axis of the aimer 426. However, various changes in visual appearance may be made in order to render an alert. For example, alternatively or in some combination a change in visual appearance may be a change in the color of the representation or in its opacity. Other forms of alert may be available, such as an overlay or alert within the main portion of the display that is not a change in appearance of the representation, audible alerts such as clicks, buzzes or beeps, or haptic feedback, may be provided. A surgeon may be provided with an option to change the nature of such alerts such that the surgeon is able to receive guidance through such alerts in a manner that suits his or her style. Furthermore, after displaying one or more alerts about the risk of collision of a guidewire with the bone fiducial 502, should the surgeon again move the aimer 426 to a pose that the surgical controller 418 determines causes the envelopes to no longer intersect, the surgical controller 418 can remove the previously-displayed collision alert(s) so that the surgeon is aware that the surgical controller 418 does not regard the current pose to be at risk of collision.

Further regarding constraints during guidance, FIG. 11D is an example video display showing computer guidance for placement of a pilot tunnel, where the surgeon has selected exit-only navigation. In the main portion of the display, the aimer 426 and field of view of the arthroscope are in a different pose than that shown in FIG. 11C. In the particular condition shown in FIG. 11D, the longitudinal central axis of the aimer 426 no longer coincides with the threads of bone fiducial 502. However, the longitudinal central axis of the aimer 426 is currently such that, were a tibial tunnel to be drilled in accordance with the longitudinal central axis, then the replacement ligament would be unable to be placed properly within it. In particular, in the pose of the longitudinal central axis shown in FIG. 11D, the exit location 705B and the path of the tunnel through the bone would result in the sum of the inter-articular distance the replacement ligament must traverse and the tibial tunnel distance itself, to exceed what would remain of the replacement ligament once it was first seated within the femoral tunnel. This may be considered an incompatibility between the femoral tunnel and the candidate tibial tunnel.

That this is the case is determined automatically by the surgical controller 418 by processing the video images to track the longitudinal central axis of the aimer 426 with respect to the three-dimensional model, and thus with respect to the tibia itself. During registration of the three-dimensional bone model, the surgical controller 418 has also registered the pose of the particular bone fiducial 502 with respect to the three-dimensional model, and thus with respect to the tibia itself. Prior to this tibial portion of the procedure, the surgical controller 418 has stored the tunnel parameters of the femoral tunnel that were determined during planning and, if the femoral tunnel has already been drilled, as a result of the drilling. These parameters may include the length of the femoral tunnel (a “first length” to be summed with others during this tibial portion of the procedure to determine a candidate traversal distance of the replacement ligament) and the exit location of the femoral tunnel into the joint. Alternatively, or in some combination, these parameters may include other measurements from which the length of the femoral tunnel may be calculated. Furthermore, as the surgical controller 418 is aware of the relative positions of the femur and tibia of the patient, and thus of the relative positions of the three-dimensional models of the femur and tibia due to registration, the surgical controller 418 is able to determine the exit location of the femoral tunnel into the joint in the frame of reference of bone fiducial 502 in the tibia. As such, the surgical controller is able to calculate the length that a replacement ligament would have to traverse through the joint from the exit location of the femoral tunnel into the joint to the exit location of the tibial tunnel into the joint that is indicated by the currently pose of the tip of the aimer 426. This may be regarded as a “third length” to be summed with others to determine the candidate traversal distance of the replacement ligament.

By tracking the longitudinal central axis of the aimer 426, the surgical controller 418 calculates an envelope of the pilot tunnel with respect to the frame of reference of the bone fiducial 502 based on the current pose of the aimer 426. It will be appreciated that a buffer zone may be included in the envelope of the pilot tunnel which corresponds to a larger diameter envelope than simply that which would be required of an actual pilot tunnel. By processing both the envelope of the pilot tunnel and the three-dimensional model of the tibia, the surgical controller 418 is able to calculate the length of the pilot tunnel within the three-dimensional model of the tibia, and thus the distance from exit location 705B to entry location 705A. This may be regarded as a “second length” to be summed with others to determine the candidate traversal distance.

By summing the distance from exit location 705B to entry location 705A and the inter-articular distance that a replacement ligament would have to traverse were a tibial tunnel to be drilled according to the current pose of aimer 426 (the current candidate tibial tunnel), the surgical controller 418 is able to determine whether this amount plus the amount the replacement ligament must traverse the femoral tunnel (the total sum being the candidate traversal distance of the replacement ligament) has an amount of difference that exceeds the overall replacement ligament length by a threshold amount, or that simply exceeds the overall replacement ligament length. Responsive to determining that this candidate traversal distance exceeds the overall replacement ligament length or the threshold, the surgical controller automatically alerts the surgeon so that the surgeon is made aware of the incompatibility of the candidate tunnel for supporting, with the femoral tunnel, a replacement ligament.

In this example, a third alert 1514 for alerting the surgeon as to the risk of incompatibility - a text alert - is shown prominently in a position on display device 414 that is adjacent the main display. The text alert 1514 provides information regarding the nature of the alert, as well as a numeral value of the extent of the incompatibility. In this example, the numerical value is a length value of 11mm, indicating to the surgeon that if a tibial tunnel were to be drilled according to the current pose of the aimer 426, the total distance the replacement ligament would have to traverse, through femoral tunnel, through the joint, and through the candidate tibial tunnel, would exceed the replacement ligament length by 11mm. Also, in this example, a fourth alert 1516 for alerting the surgeon as to the risk of collision – a graphical alert – is shown in the form of the representation of the longitudinal central axis of the aimer 426 changing in visual appearance. In this way, the surgeon when navigating within the surgical site can be provided with visual feedback as to the appropriateness of a particular position of aimer 426. In this example, the visual appearance change is a change in the line pattern of the representation of the longitudinal central axis of the aimer 426. However, various changes in visual appearance may be made in order to render an alert. For example, alternatively or in some combination a change in visual appearance may be a change in the color of the representation or in its opacity. Other forms of alert may be available, such as an overlay or alert within the main portion of the display that is not a change in appearance of the representation, audible alerts such as clicks, buzzes or beeps, or haptic feedback, may be provided. A surgeon may be provided with an option to change the nature of such alerts such that the surgeon is able to receive guidance through such alerts in a manner that suits his or her style. Furthermore, after displaying one or more alerts about the risk of incompatibility of the candidate tibial tunnel, should the surgeon again move the aimer 426 to a pose that the surgical controller 418 determines causes the incompatibility to no longer hold, the surgical controller 418 can remove the incompatibility alert(s) so that the surgeon is aware that the surgical controller 418 does not regard the now-current pose of the aimer 426 to indicate a candidate tibial tunnel that is at risk of incompatibility with the femoral tunnel for together supporting the replacement ligament.

In this example, a fifth alert 1518 for alerting the surgeon as to the risk of incompatibility - a text alert - is shown prominently in a position on display device 414 that is adjacent the main display. The kind of incompatibility indicated by fifth alert 1518 is due to the risk of impingement. The text alert 1518 provides information regarding the nature of the alert, as well as a numeral value of the extent of this incompatibility. In this example, the numerical value is a percentage amount of 17%, indicating to the surgeon that if a tibial tunnel were to be drilled according to the current pose of the aimer 426, the portion of the replacement ligament passing through the joint from the femur to the tibia would be impinged by a percentage amount of 17%. That is, that the replacement ligament and the bones would interfere such that 17% of the ligament would coincide with at least one of the bones. As impingement, by bones, of the portion of the replacement ligament meant to pass from femur to tibia is to be avoided, or at least that a threshold level of impingement – like to be a small level of impingement – not be exceeded, this alert provides the surgeon with guidance enabling the surgeon to choose a different exit location 705B thereby to avoid the impingement.

That incompatibility due to impingement that would occur if the current exit location 705B were used, is determined automatically by the surgical controller 418 by processing the video images to track the tip of the aimer 426 with respect to the three-dimensional model, and thus with respect to the tibia itself. During registration of the three-dimensional bone model, the surgical controller 418 has also registered the pose of the particular bone fiducial 502 with respect to the three-dimensional model, and thus with respect to the tibia itself. Prior to this tibial portion of the procedure, the surgical controller 418 has stored the tunnel parameters of the femoral tunnel that were determined during planning and, if the femoral tunnel has already been drilled, as a result of the drilling. These parameters include the length of the femoral tunnel and the exit location of the femoral tunnel into the joint. Furthermore, as the surgical controller 418 is aware of the relative positions of the femur and tibia of the patient, and thus of the relative positions of the three-dimensional models of the femur and tibia due to registration, the surgical controller 418 is able to determine the exit location of the femoral tunnel into the joint in the frame of reference of bone fiducial 502 in the tibia. By tracking the tip of the aimer 426 the surgical controller 418 is able to determine the exit location of the candidate tibial tunnel into the joint. As such, the surgical controller 418 is able to calculate the candidate path through the joint that a replacement ligament would have to traverse from the exit location of the femoral tunnel into the joint over to the exit location of the tibial tunnel into the joint that is indicated by the current position of the tip of the aimer 426. With this data, and with data corresponding to the thickness of the replacement ligament provided by the surgeon to the surgical controller 418, the surgical controller 418 is able to calculate an envelope of the candidate path of the replacement ligament through the joint. The surgical controller 418 is thereby able to calculate whether the envelope of the candidate path of the replacement ligament through the joint coincides with either the three-dimensional model of the femur or the three-dimensional model of the tibia and, if so, by how much. Responsive to determining that the envelope of the candidate path of the replacement through the joint coincides with the models, the surgical controller 418 automatically alerts the surgeon so that the surgeon is made aware of the incompatibility of the candidate tunnel for supporting, with the femoral tunnel, a replacement ligament.

Also, in this example, the fourth alert 1516 – the graphical alert – is not changed or compounded due to there being two incompatibilities (exceeded replacement ligament length; replacement ligament impingement risk). However, a surgeon may be provided with the option to change the graphical alert responsive to multiple incompatibilities or other constraint violations being determined by surgical controller 418. Furthermore, after displaying one or more alerts about the risk of incompatibility of the candidate tibial tunnel, should the surgeon again move the aimer 426 to a pose that the surgical controller 418 determines causes all incompatibilities to no longer hold, the surgical controller 418 can remove the incompatibility alert(s) so that the surgeon is aware that the surgical controller 418 does not regard the now-current pose of the aimer 426 to indicate a candidate tibial tunnel that is at risk of incompatibility with the femoral tunnel for together supporting the replacement ligament.

Further regarding constraints during guidance, FIG. 11E is an example video display showing computer guidance for placement of a pilot tunnel, where the surgeon has selected exit-only navigation. In the main portion of the display, the aimer 426 and field of view of the arthroscope are in a different pose than that shown in FIG. 11D, but the location of the tip of the aimer 426 has not changed. Calculating that the incompatibility risk due to the tunnel length being too long no longer holds, the surgical controller 418 removes the previously-displayed text alert 1514. However, text alert 1518 remains displayed since the impingement risk has not changed, and graphical alert 1516 remains displayed because the surgical controller 418 continues to calculate an incompatibility.

The surgical controller 418 may process the data about the femoral tunnel, the candidate tibial tunnel, the candidate path through the joint of a replacement ligament, the three-dimensional bone models, and their relative positions in respective coordinate systems, to determine other incompatibilities and to display corresponding alerts. For example, the surgical controller 418 may calculate that the angle of a candidate tibial tunnel with the tibial plateau is outside of angle constraints established either by the surgeon and provided to the surgical controller 418 as a surgical parameter, or in accordance with the literature on best practices for an ACL repair and provided to the surgical controller 418 as a surgical parameter. Furthermore, a minimum length parameter for establishing a minimum length of tunnel and/or of overall replacement ligament traversal distance through femur, joint and tibia, may be maintained by the surgical controller 418 to provide guidance to ensure that the current drill axis position would not result in too-short a tunnel. In this manner, alerts may be presented based on a tunnel being too short as well as a tunnel being too long, such that the candidate traversal distance is determined to be less than a preset value provided by the surgeon to the surgical controller 418 as a minimum distance or is determined to be greater than a present value provided by the surgeon to the surgical controller 418 as a maximum distance. Such alerts may be presented in the same manner – such as a same color – or in a different manner – such as in different colors for too short (less than a minimum preset value) and too long (greater than a maximum preset value), depending on the configuration and/or a user preference setting stored by the surgical controller 418.

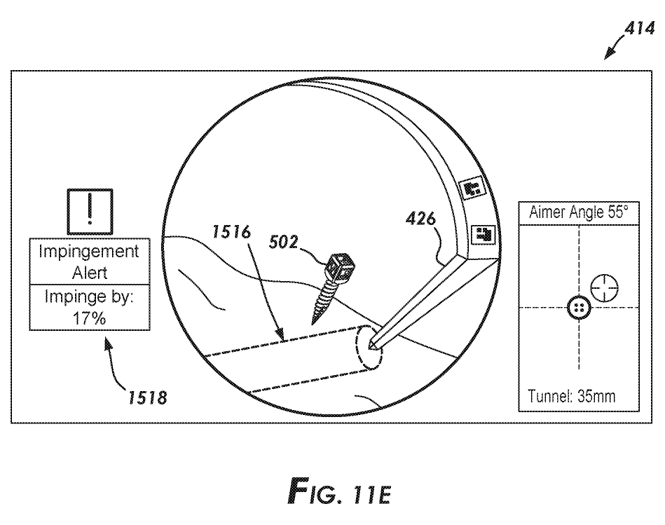

With the surgeon being guided using information displayed on display device 414 as to positioning of the drill axis of a drill wire 424 (or “guidewire”) based on tracking of an aimer 426 or a drill wire 424 itself, the surgeon may also be provided with information and options for establishing parameters, by tablet computer 614. FIG. 12 is a display screen of tablet computer 614 providing tibial tunnel settings, measurements, and guidewire capture options based on tracking of the instrument as described herein in connection with FIGS. 11A-E. In particular, as the surgeon hones in, using the surgical guidance described in connection with FIGS. 11A-E, on a drill axis being tracked by the surgical controller 418, the surgeon may select various options, such as Elbow/Tip options for an aimer 426, the navigation method, in this example set at Trajectory but also offering Exit-Only as an option, the tunnel diameter, in this example set at 9mm, the aimer angle, in this example set at 55 degrees, the minimum tibial tunnel length, in this example set at 30mm. Also displayed are tunnel length and angle parameters for the planned tunnel, set respectively at 33mm and 55 degrees. Also displayed are tunnel length and angle parameters for the previously planned tunnel, set respectively at 36mm and 55 degrees. It will be appreciated that the surgeon may capture a new guidewire position, thus updating the planned tunnel parameters and changing the previously planned tunnel parameters to what have before been the planned tunnel parameters. The surgeon may select a Capture guidewire position user interface button 660, which instructs surgical controller 418 to capture the drill axis corresponding to the current pose of the aimer 426, so that the surgical controller can calculate the length of the candidate tunnel and its angle with the plateau. The surgeon may make other selections using respective user interface buttons to Capture a new planned tunnel, reset to the original planned tunnel, or go back to a previous display screen.

In another example, a navigation method or guidance display approach may be provided, either exclusively or as an alternative to the Tip or Trajectory navigation options, by the surgical controller 418 in which, in a first stage of navigation only guidance for the tip of the aimer is provided, for example by displaying just the crosshair for navigation of the tip. Then, once the surgical controller 418 detects through processing of the image frames that the tip of the aimer has been placed by the surgeon at, or in appropriate proximity to, the planned tibial tunnel exit location, guidance for the entry location and/or the entire trajectory is displayed, for example by fading-in or otherwise displaying the crosshair for navigation of the entry location/trajectory. In this way, the surgeon may be guided to begin with first placing the tip and then, only once this has been done, then proceed with placing the bullet of the aimer while maintaining the tip in place.

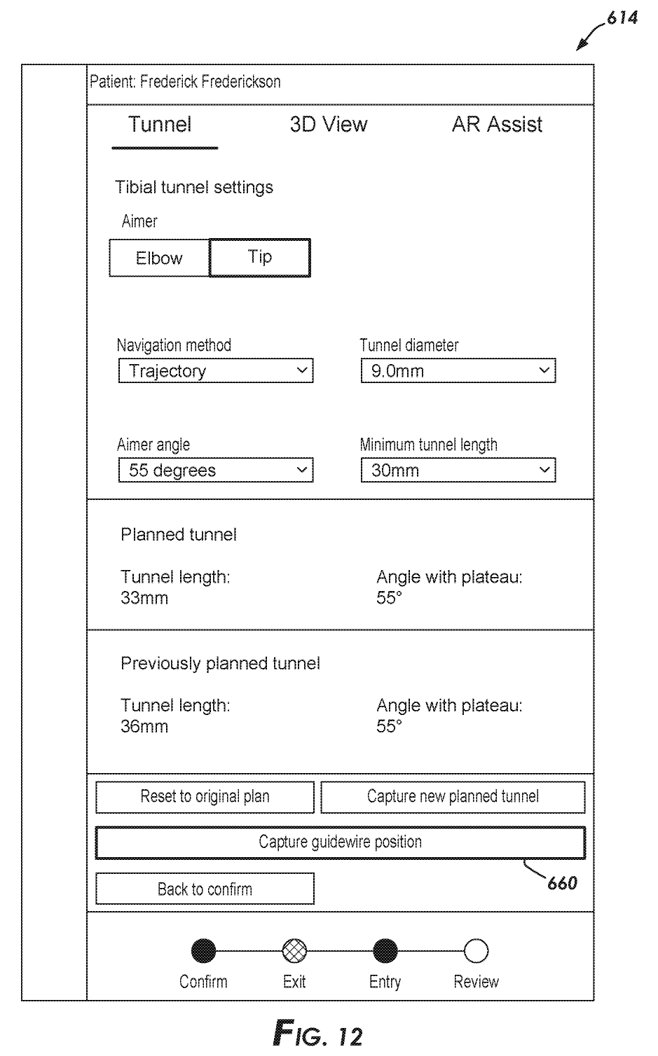

FIG. 13 is a display screen of tablet computer 614 providing a view of the three-dimensional model of the tibia 704, showing both the planned tunnel trajectory and the captured guidewire position in relation to one another, as well as other information described herein in connection with other display screens.

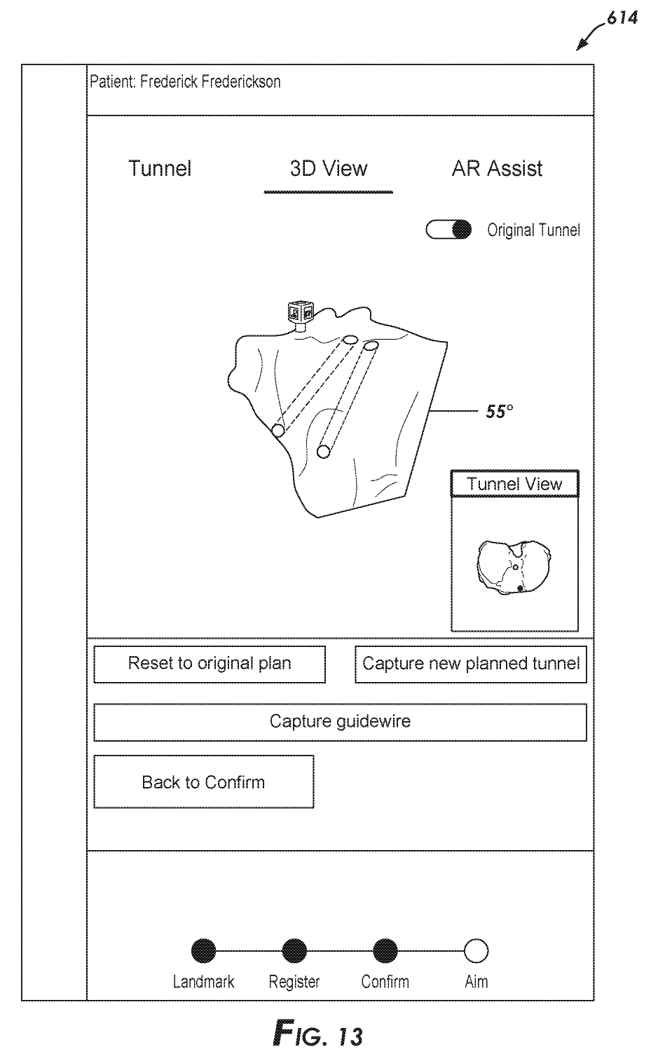

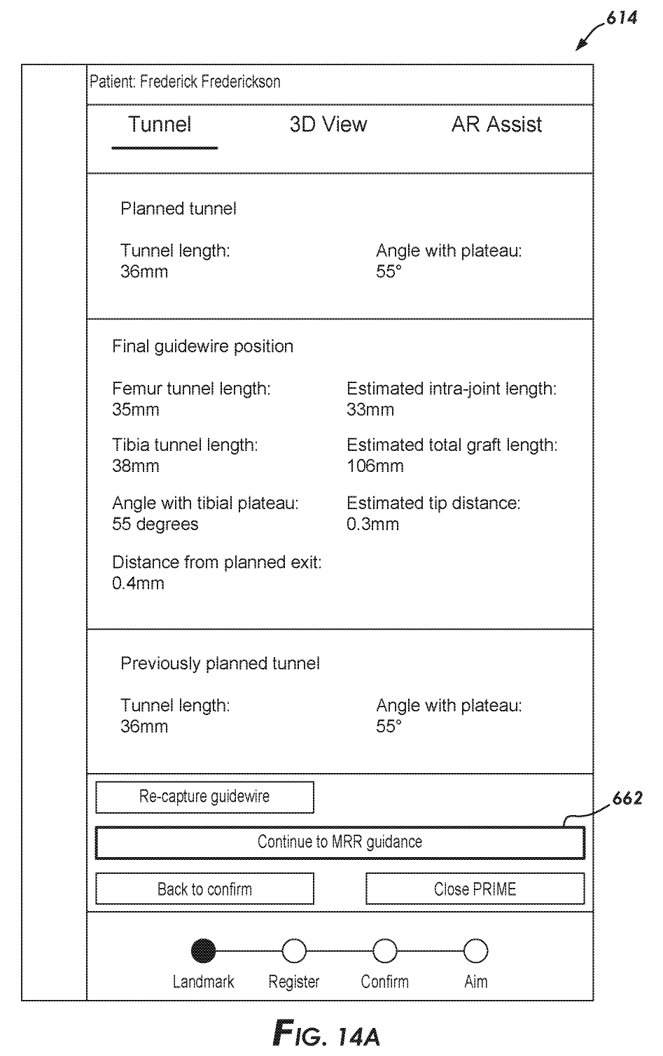

FIG. 14A is a display screen of tablet computer 614 providing a planned tibial tunnel overview based on a captured guidewire position that is considered by the surgeon to be within all constraints and useful for drilling. Shown are parameters including the femur tunnel length, in this example 35mm, the tibia tunnel length, in this example 38mm, the estimated intra-joint graft length, in this example 33mm, and the estimated total graft length, in this example 106mm being the sim of the femur tunnel length, the tibia tunnel length, and the intra-joint graft length. Also shown are the angle of the tibial tunnel with the tibial plateau, in this example 55 degrees, the estimated tip distance, in this example 0.3mm, and the distance of the final exit location into the joint of the tibial tunnel, from the planned exit, in this example, 0.4mm. Information about the previously planned tunnel is also shown. The surgeon is provided with user interface buttons for re-capturing a guidewire, going back to confirm, and closing the application altogether.

Also shown in FIG. 14 is a user interface button 662 for enabling the surgeon to continue to planning and guidance for meniscal root repair (MRR). It will be appreciated that, while operating within the surgical site for ACL repair, it may be useful to also conduct a MRR, but that doing so may require that one or more additional tunnels be drilled into the tibia. The MRR guidance described herein uses the data stored by the surgical controller about the final tibial tunnel for ACL repair – the first tibial tunnel – to be available to the surgical controller 418 to serve to constrain the available positions of the tunnel for MRR repair – the second tibial tunnel. In this way, the second tibial tunnel trajectory can be planned such that it is appropriately positioned for the MRR itself, but also such that it does not intersect with the first tibial tunnel, and furthermore does not converge on the tibial tunnel in such a manner as to create a bone bridge between the first and second tunnel that is too small, and that is therefore at a risk of collapsing.

FIG. 14B is an example video display presented on display device 414 showing computer guidance for placement of an MRR tunnel. Displayed in a solid line 1522 is the drill axis for the guidewire according to a different pose of aimer 426. Also displayed are surgical parameters relating to the tibial tunnel for ACL repair. It will be appreciated that, for MRR guidance, a different aimer of the same or a different kind may be used for guidewire positioning for MRR.

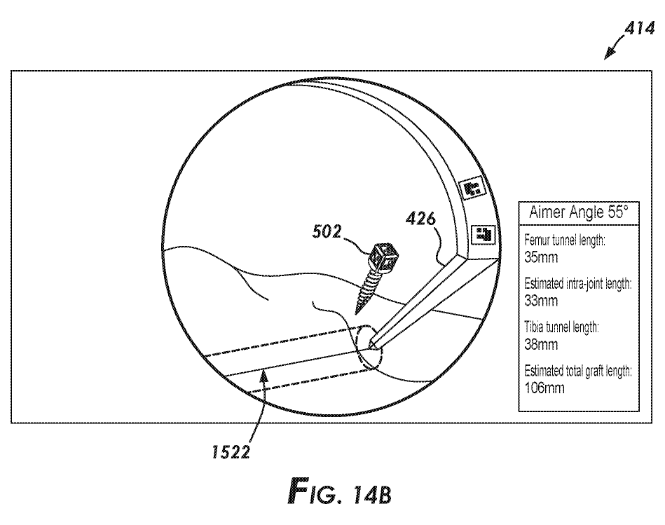

FIG. 15 is a display screen of tablet computer 614 providing computer guidance for placement of an MRR tunnel, pursuant to a pose of guidewire – and thus the trajectory of the drill axis - having been captured based on the current pose of the aimer 426. In the main panel is shown the planned ACL tunnel as well as the candidate MRR tunnel that would be formed in accordance with the captured guidewire position. While the surgeon is being guided with navigation using screens of display device 414, the surgeon can also look to display screens on tablet computer 614 to provide guidance with respect to a more global view of the three-dimensional model of the tibia. The surgeon is thus able to visually inspect the relationship between the final tibial ACL tunnel and the candidate MRR tunnel being decided-upon. It will be appreciated that preoperative planning for the MRR tunnel itself may not be done, such that the surgeon may be first determining candidate MRR tunnel trajectories intraoperatively. In such a case, a planned MRR tunnel will not, at first, be available to the surgical controller 418 for constraining the scope of MRR tunnel trajectories. However, once the surgeon intraoperatively captures a guidewire position, previous captures can be presented on one or both of the tablet computer 614 and the display device 414 so that the surgeon is able to compare a current guidewire capture to at least the previous capture.

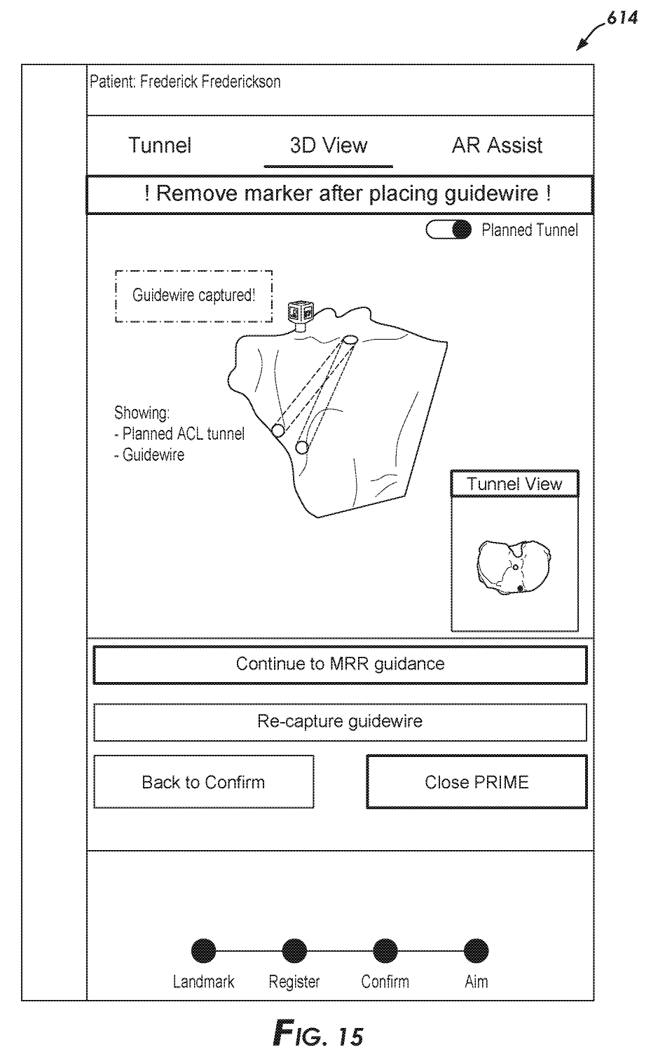

FIG. 16 is a display screen of tablet computer 614 providing computer guidance for placement of the MRR tunnel, enabling selection of options such as aimer type, in this example an MRR aimer, aimer angle, in this example 55 degrees, a minimum bone bridge between tunnels, in this example 5mm, and an MRR tunnel diameter, in this example 2.4mm. These parameters may be used by surgical controller 418 to define, along with the tracking by the surgical controller of the drill axis of the instrument (an MRR aimer, a drill wire, or some other instrument defining a drill axis) as indicated by the longitudinal axis of the instrument, a candidate MRR tunnel – a candidate second tunnel - in the tibia. This data, as well as data stored by the surgical controller defining the tibial ACL tunnel – the first tunnel - may be used by the surgical controller 418 to calculate at least one value indicative of a risk of convergence, in the tibia, of this candidate second tunnel with the first tunnel. More particularly, by tracking the longitudinal central axis of the aimer 426, and with consideration of at least the surgical parameter representing the diameter of the candidate MRR tunnel, the surgical controller 418 calculates an envelope of the candidate MRR tunnel with respect to the frame of reference of the bone fiducial 502 based on the current pose of the aimer 426. It will be appreciated that a buffer zone may be included in the envelope of the candidate MRR tunnel which corresponds to a larger diameter envelope than simply that which would be required of an actual MRR tunnel. By processing both the envelope of the candidate MRR tunnel and the envelope of the ACL tunnel, the surgical controller 418 is able to calculate the one or more values indicative of the risk of these two envelopes converging, and display surgical guidance based on the one or more values. For example, to calculate a value indicative of the two envelopes converging, the surgical controller 418 may measure a distance between the closest points along the envelopes, corresponding to the closest points along the ACL tunnel and the candidate MRR tunnel. The surgical controller 418 may thereafter compare this measured distance to determine whether it is below a threshold distance. For example, the threshold distance may be a minimum bone bridge thickness – shown for example in the dropdown of FIG. 16 as being set at 5mm. If the distance between the closest points along the envelopes is lower than 5mm, the surgical controller 418 may cause to be displayed, on display device 414 and/or display device of tablet computer 614, surgical guidance information in the form of an alert that indicates to the surgeon that the bone bridge between the ACL tunnel and the candidate MRR tunnel, if it were to be drilled, would be below the threshold. Furthermore, the surgical controller 418 may provide guidance information in the form of an actual bone bridge thickness amount corresponding to the distance between the closest points, so that the surgeon is able to have confirmation both that the bone bridge thickness between the ACL tunnel and a particular candidate MRR tunnel meets or exceeds the minimum and that the bone bridge thickness between the ACL tunnel and a different particular candidate MRR tunnel is less than the minimum.

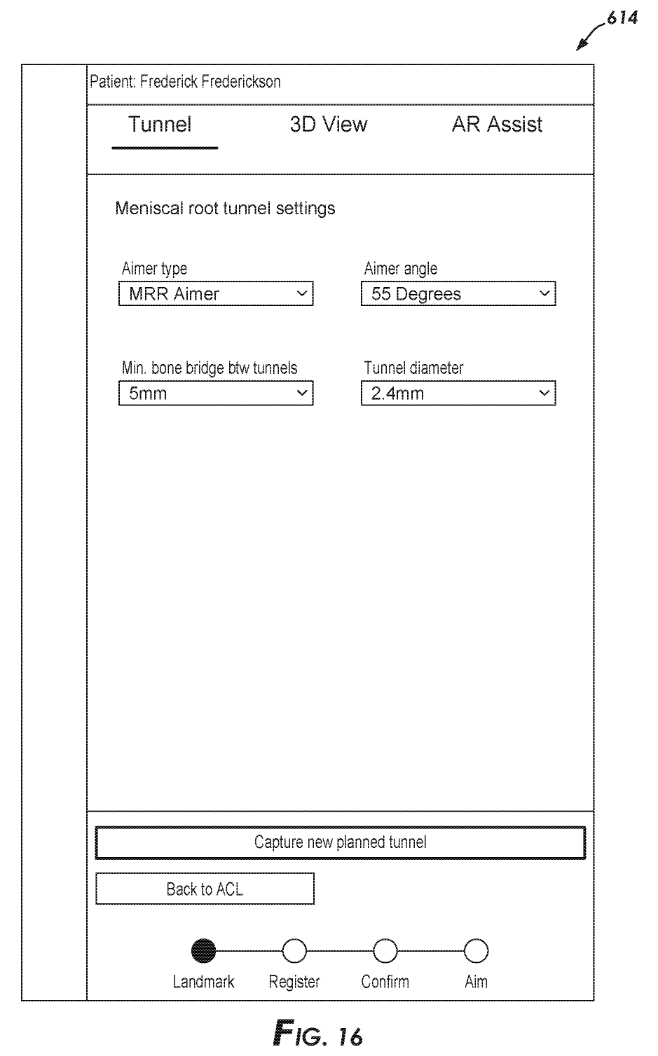

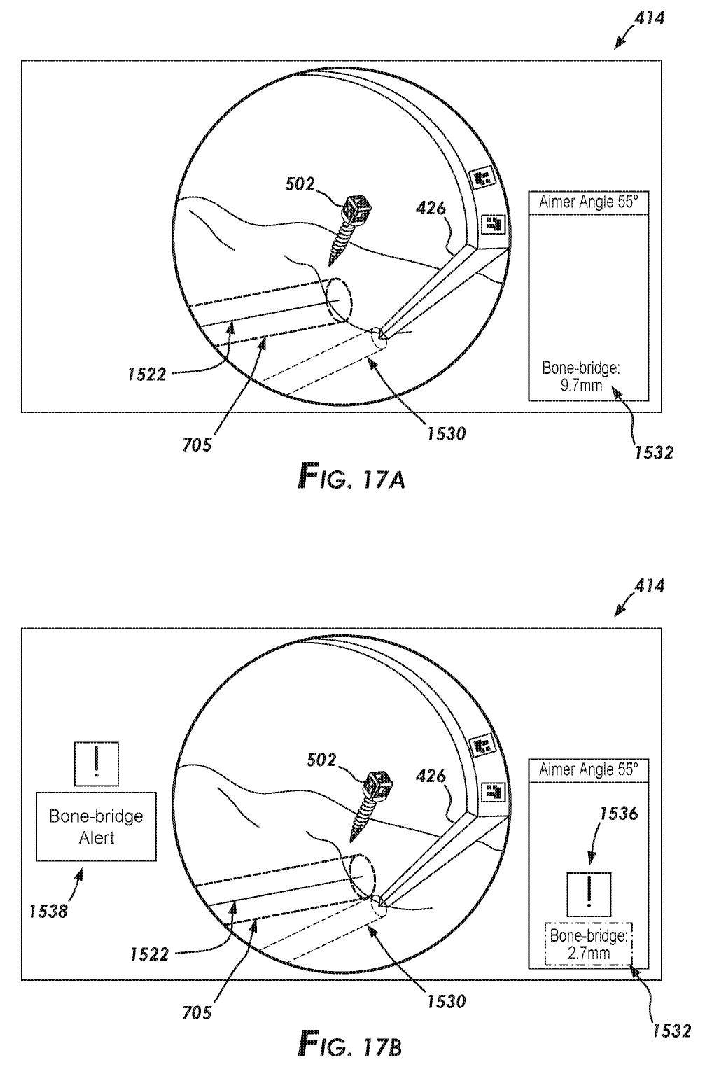

FIG. 17A is an example video display of display device 414 showing computer guidance for placement of an MRR tunnel. In the main portion of the display is a portion of the bone in the video images as captured by the arthroscope 408, a representation of the envelope of the final ACL tibial tunnel path 705, and a representation of the envelope of the candidate MRR tunnel path 1530 generated by the surgical controller 418 based on the tracking of the tip and pose of the aimer 426, and thus its longitudinal central axis, and the MRR tunnel diameter parameter (FIG. 16). Displayed outside of the main display is the inter-tunnel bone bridge thickness value 1532, in this example 9.7mm.

FIG. 17B is an example video display of display device 414 showing computer guidance for placement of an MRR tunnel. In the main portion of the display is a portion of the bone in the video images as captured by the arthroscope 408, a representation of the envelope of the final ACL tibial tunnel path 705, and a representation of the envelope of the candidate MRR tunnel path 1530 generated by the surgical controller 418 based on the tracking of the tip and pose of the aimer 426, and thus its longitudinal central axis, and the MRR tunnel diameter parameter (FIG. 16). As compared with the conditions shown in FIG. 17A, the tip and pose of aimer 426 are such that the envelope of the candidate MRR tunnel 1530 is closer to the envelope of the final ACL tunnel 705. In this condition, the surgical controller 418 has calculated that the closest points of the two envelopes are less than the threshold bone bridge distance of 5mm apart. Displayed outside of the main display is the inter-tunnel bone bridge thickness value 1532, in this example 2.7mm. Because this bone bridge thickness value, indicative of convergence, is below the threshold bone bridge distance, the surgical controller 418 causes to be displayed on display device 414 at least one alert as a form of surgical guidance information for use by the surgeon. In this example, the at least one alert includes an alert 1536 – a combined text and visual alert – that frames the bone bridge thickness value 1532 with a box to draw the attention of the surgeon if he or she is watching that value while honing in on a pose of the aimer 426. In this example, another alert 1538 – a text alert – is displayed prominently on the opposite side of the main display to raise the surgeon’s awareness in an additional way, that the inter-tunnel bone bridge threshold has not been met with the current position of the candidate MRR tunnel.

The surgeon when navigating within the surgical site may additionally be provided with visual feedback as to the appropriateness of a particular position of aimer 426 for positioning an MRR tunnel. The visual appearance change may be a change in the line pattern of the representation of the longitudinal central axis of the aimer 426. However, various changes in visual appearance may be made in order to render an alert. For example, alternatively or in some combination a change in visual appearance may be a change in the color of the representation or in its opacity. Other forms of alert may be available, such as an overlay or alert within the main portion of the display that is not a change in appearance of the representation, audible alerts such as clicks, buzzes or beeps, or haptic feedback, may be provided. A surgeon may be provided with an option to change the nature of such alerts such that the surgeon is able to receive guidance through such alerts in a manner that suits his or her style. Furthermore, after displaying one or more alerts about the risk of convergence of the candidate MRR tunnel with the final ACL tibial tunnel, should the surgeon again move the aimer 426 to a pose that the surgical controller 418 determines causes their envelopes to no longer intersect, the surgical controller 418 can remove the convergence/bone bridge alert(s) so that the surgeon is aware that the surgical controller 418 does not regard the current pose to be at risk of tunnel convergence.

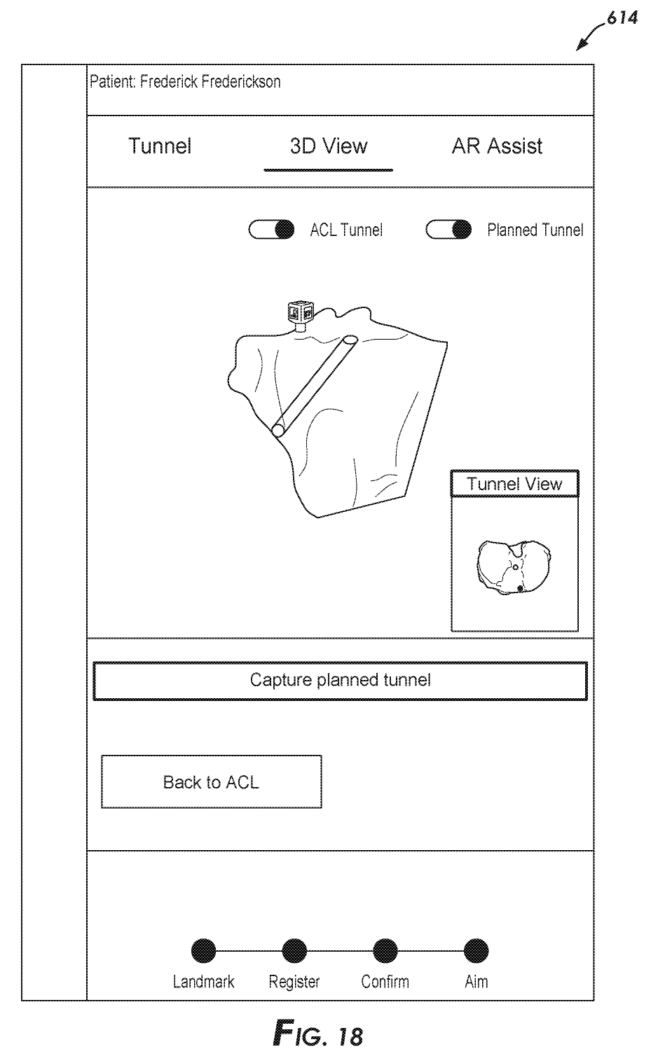

The surgeon is able to consult with the display presented on tablet computer 614 to provide a global view of a captured guidewire position based on a current position of the aimer 426, and thus a display with respect to the three-dimensional model of the tibia of both the ACL tunnel and the planned MRR tunnel. FIG. 18 is a display screen of tablet computer 614 providing a view of the three-dimensional model of the tibia 704, showing the ACL tunnel prior to capture of a guidewire position for a planned tunnel, as well as other information described herein in connection with other display screens.

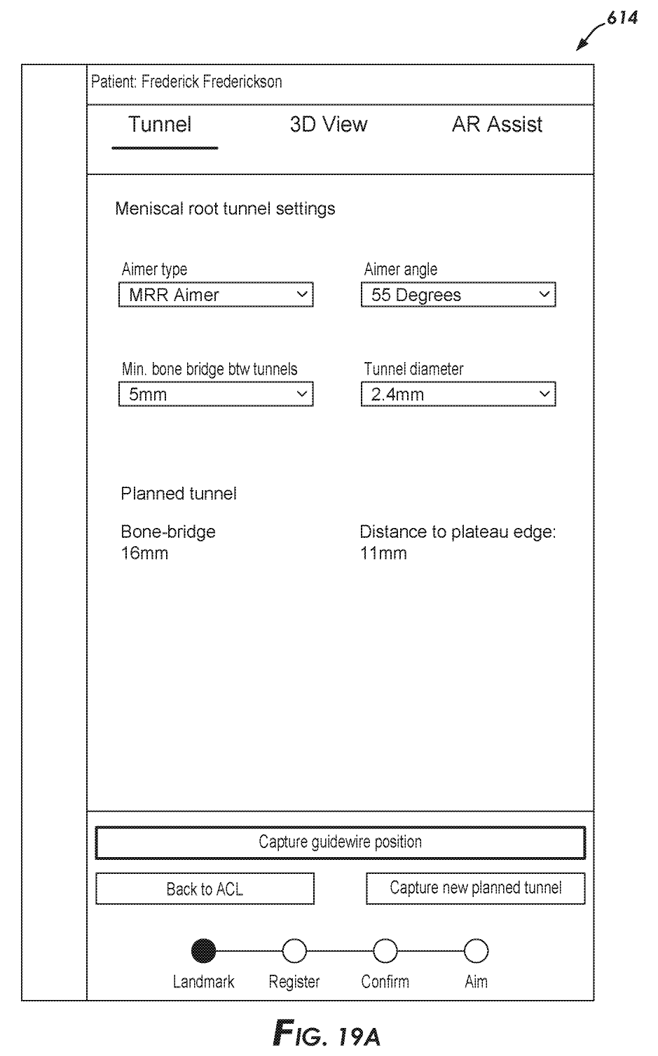

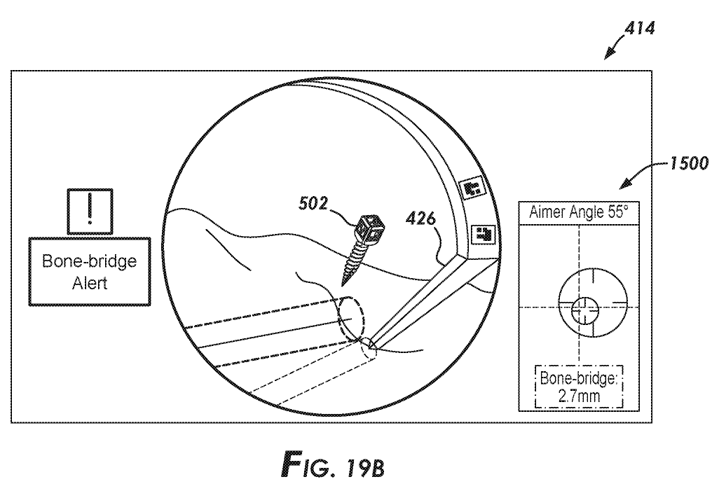

FIG. 19A is a display screen of tablet computer 614 providing a view of the surgical parameters and a Capture guidewire position button that can be selected by a surgeon while the surgeon is navigating in the surgical site. FIG. 19B is an example video display of display device 414 showing computer guidance for placement of an MRR tunnel that may be presented to the surgeon while tablet computer 614 is presenting the content shown in FIG. 19A, including the selectable Capture guidewire position button. If the surgeon were to select the Capture guidewire position button using table computer 614, the surgical controller 418 would, in response, capture a new Planned tunnel. It will be noted that in this example the new Planned tunnel position would have triggered a bone bridge alert due to the minimum bone bridge threshold not having been met, but the surgeon is able to re-capture a guidewire position by first re-positioning the aimer 426 and re-selecting a capture guidewire option.

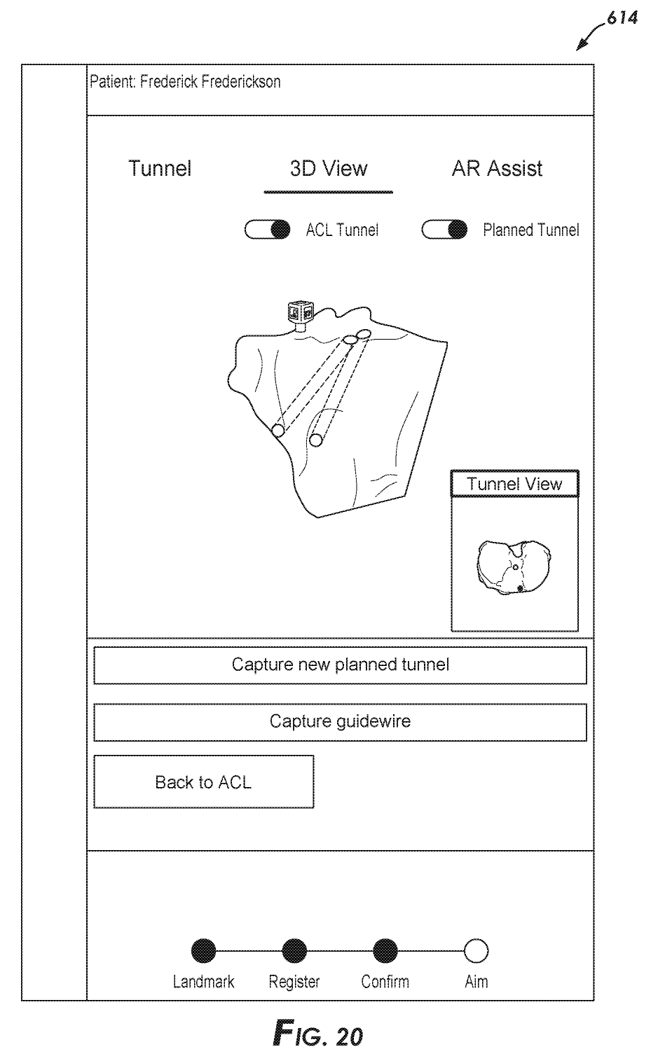

FIG. 20 is a display screen of tablet computer 614 providing a view of the three-dimensional model of the tibia 704, showing both the planned tunnel trajectory and the ACL tunnel positions in relation to one another, as well as other information described herein in connection with other display screens. The surgeon is able to capture a new guidewire position and to confirm that as a captured new planned tunnel, from this display screen.

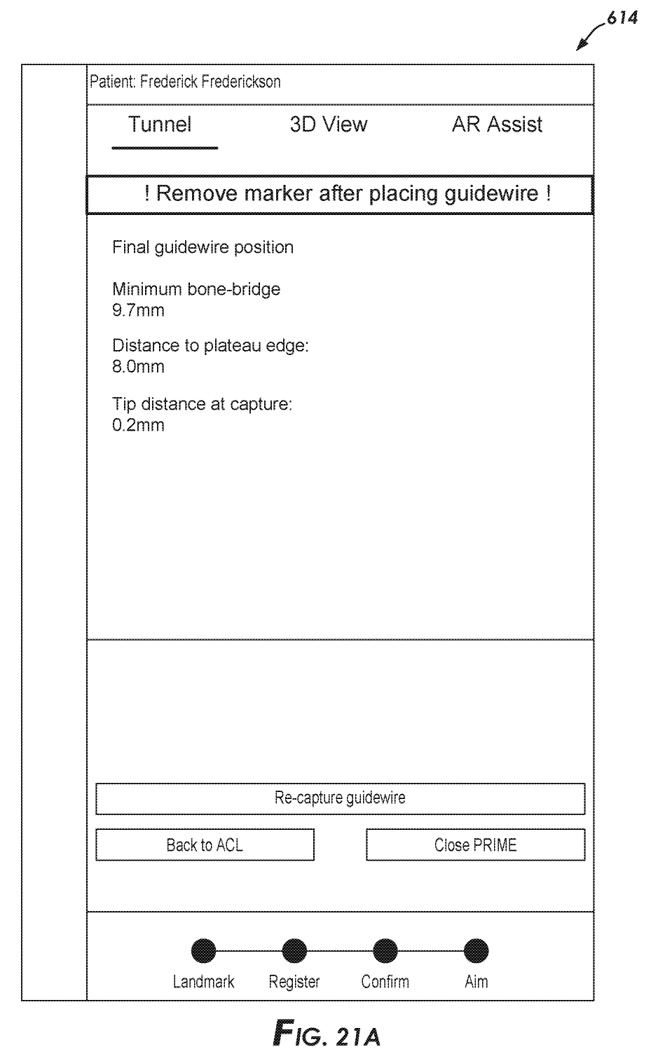

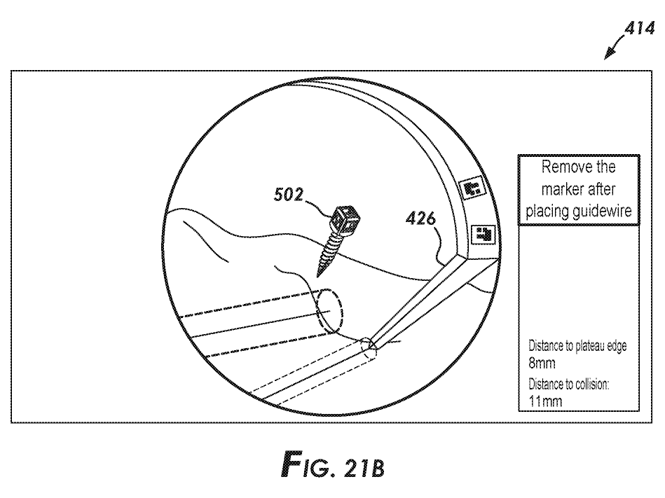

FIG. 21A is a display screen of tablet computer 614 providing surgical parameters regarding the final guidewire position captured, including a minimum bone-bridge, in this example 9.7mm. It will be noted that the minimum bone-bridge is not, in this display screen, the minimum threshold, but is the minimum distance measured by the surgical controller between the envelope of the final ACL tunnel and the envelope of the MRR tunnel at the final guidewire position. Also shown are other surgical parameters measured by the surgical controller 418 based on the final guidewire position, including the distance to plateau edge, in this example 8.0mm, and the tip distance at capture, in this example 0.2mm. The surgeon is provided with the option of Re-capturing a guidewire position based on the pose of the aimer 426 at the time of selecting the Re-capture guidewire user interface button in FIG. 21A. FIG. 21B is an example video display of display device 414 showing computer guidance for placement of the MRR tunnel that may be presented to the surgeon while tablet computer 614 is presenting the content shown in FIG. 21A.

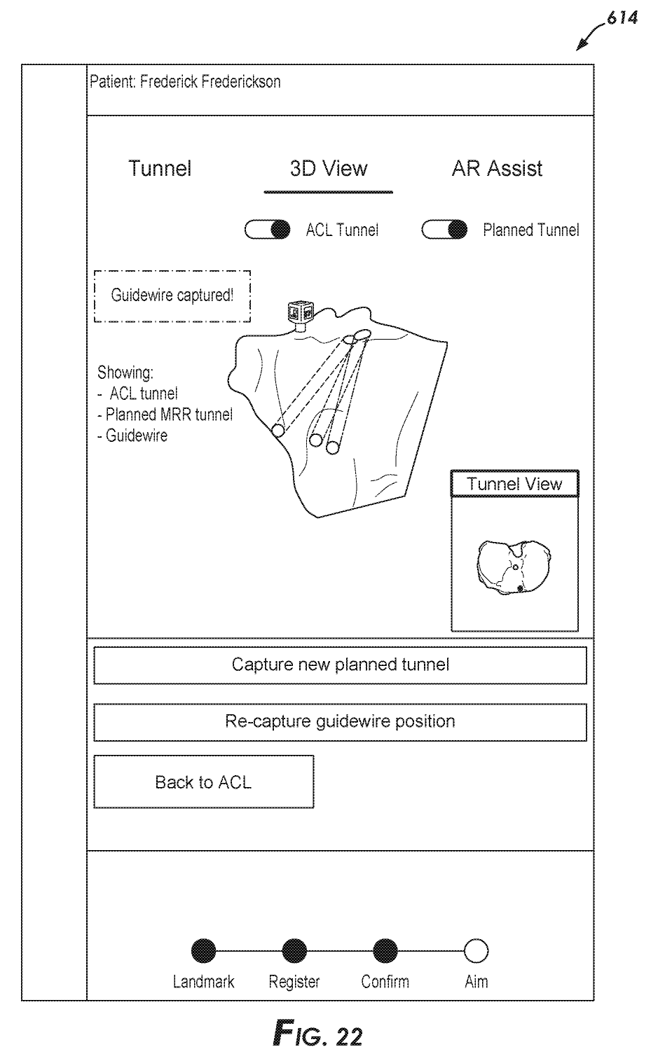

FIG. 22 is a display screen of tablet computer 614 providing a view of the three-dimensional model of the tibia 704, along with representations of each of the final ACL tunnel, the planned (or previous) MRR tunnel, and a tunnel that would be formed were the guidewire position recently captured selected by the surgeon as the new planned tunnel. The display screen provides user interface options for toggling presentation of the ACL tunnel and the planned tunnel, as well provides information available via other screens, as described herein.

### TUNNEL CREATION

With the revised-tunnel path 705 selected, the next step in the example method is creation of the actual tunnel. In most cases, creating the tunnel is a multistep process involving drilling an initial or pilot tunnel using a drill wire (e.g., drill wire 424 (FIG. 4)), and then using the drill wire as guide wire for one or more reamers to increase the diameter of the pilot tunnel to form the full-diameter actual tunnel in the bone. In some cases the actual tunnel in the femur has a counterbore associated with the intercondylar notch, and the actual tunnel in the tibia has a counterbore associated with the tibial plateau, to accommodate the width of replacement ligament, and in such cases an additional reamer may be used to create such counterbores.

The discussion with respect to FIGS. 11A-E-E and the graphic 1500 assumed use of the aimer 426 to locate the drill wire 424 (FIG. 4) prior to drilling the pilot hole. Whether or not the drill wire is disposed within the aimer 426 during the alignment process, the longitudinal central axis of the aimer 426 nevertheless represents the expected drill axis once drilling of the pilot tunnel begins. In other cases the aimer 426 may be omitted, and the drill wire 424 itself may be tracked. That is, the drill wire 424 may include a wire fiducial having one more machine readable patterns from which the surgical controller 418 can determine the location of the distal end of the drill wire 424 and the orientation of the longitudinal central axis of the drill wire (at least proximate to the bone within the surgical site). Thus, the discussion to this point assuming use of the aimer 426 as the mechanism for enabling placement and orientation of the drill wire prior to drilling shall not be read as limitation of the claims. For example, the surgical controller is tracking the drill axis of an instrument, whether the instrument be an aimer, the drill wire itself, or another instrument providing a drill axis, with respect to the bone.

Returning to the aimer example, however, once the aimer 426 is aligned with the revised-tunnel path 705, and the surgeon is satisfied that there are no incompatibilities or risk of collision, drilling of the pilot tunnel commences. If the drill wire 424 (FIG. 4) is not already within the aimer 426, the surgeon telescopes the drill wire 424 within the aimer 426. Drilling may involve the surgeon holding the aimer 426 in the desired orientation as shown by the graphic 1500, and providing rotational energy to the drill wire 424, such as by an external drill assembly. Once the drill wire enters the bone, the drill wire 424 drills through the bone in a straight line, and ultimately exits the bone on the far side. For an inside-out procedure, the drill wire also exits the skin on the outside portion of the leg. Once the drill wire 424 completes the pilot tunnel, with the aimer 426 still telescoped over the drill wire 424, placement of the pilot tunnel relative to the revised-tunnel path 705 may be analyzed.

In situations in which a particular selected drill wire trajectory may collide with a bone marker, a surgeon may choose to partially drill the tunnel towards the bone marker using surgical guidance anchored by the bone marker, but stop short before the drill wire collides with the bone marker. With the tunnel having been partially drilled, its direction may be generally established such that actual surgical guidance for continued drilling from this point may be less important. As such, prior to proceeding along the remainder of the drilling trajectory, the surgeon may choose to first remove the bone marker such that when thereafter continuing forward with the drilling along the established direction, such a collision is avoided.

### TUNNEL PLACEMENT ANALYSIS

In accordance with example methods and systems, prior to using the reamer(s) to create the full diameter tunnel through the bone, the surgical controller 418 (FIG. 4) may provide information to the surgeon regarding the relationship between the pilot tunnel and the revised-tunnel path 705 to enable the surgeon to determine whether the pilot tunnel should be used as the guide to create the actual tunnel through the bone. Examples of the surgical controller 418 providing such information is described in Quist et al.

It will be appreciated that some or all of the content displayed on the tablet computer 614 may instead, or additionally, be displayed on the display device 414. Furthermore, content described as displayed on any of the display devices disclosed herein, and operations accessible to a user from any of the computing devices disclosed herein, may be consolidated into a single display/computing device, provided the single display/computing device is suitable in size and quality for providing sufficient surgical guidance and sufficient usability. Similarly, content described as displayed on another of the display device disclosed herein, and operations accessible to a user from any of the computing devices disclosed herein, may be spread amongst multiple display/computing devices. Display and provision of user interface options may be done using any number of computing and display devices under the control of surgical controller 418.

Alternatively or in some combination, the surgical controller 418 may be caused to generate an output, such as a report in PDF (portable document format) format containing details of the tunnel placement and any other parameters and factors, so that this information may be stored and/or studied.

### SOFTWARE AND HARDWARE

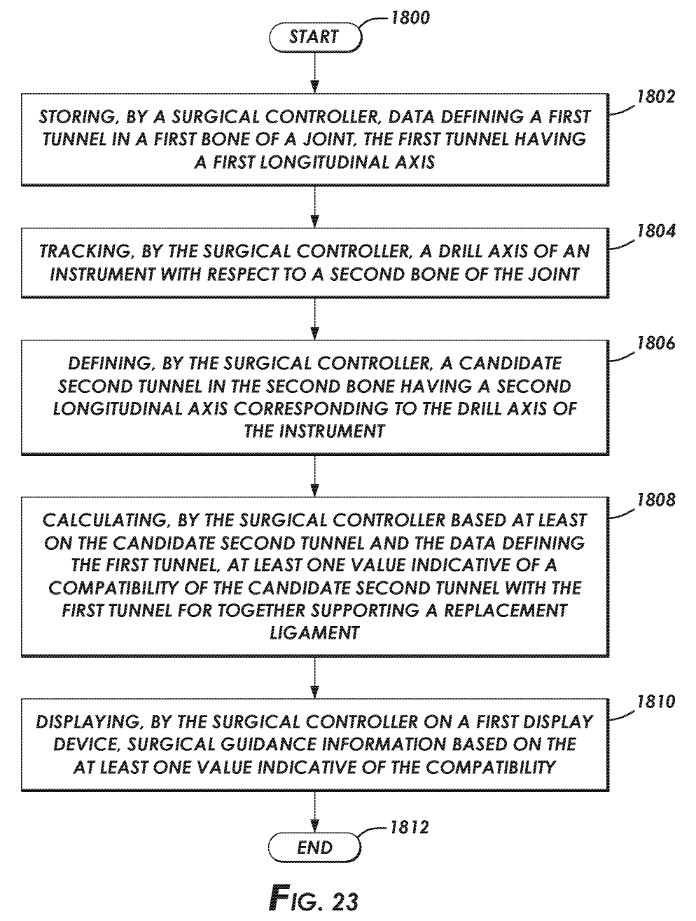

FIG. 23 shows a method in accordance with at least some embodiments. In particular, the method starts (block 1800) and comprises: storing, by a surgical controller, data defining a first tunnel in a first bone of a joint, the first tunnel having a first longitudinal axis (block 1802); tracking, by the surgical controller, a drill axis of an instrument with respect to a second bone of the joint (block 1804); defining, by the surgical controller, a candidate second tunnel in the second bone having a second longitudinal axis corresponding to the drill axis of the instrument (block 1806); calculating, by the surgical controller based at least on the candidate second tunnel and the data defining the first tunnel, at least one value indicative of a compatibility of the candidate second tunnel with the first tunnel for together supporting a replacement ligament (block 1808); and displaying, by the surgical controller on a first display device, surgical guidance information based on the at least one value indicative of the compatibility (block 1810). Thereafter, the method ends (block 1812). The example method may be implemented by computer instructions executed with the at least one processor of a computer system, such as the surgical controller 418 (FIG. 4).

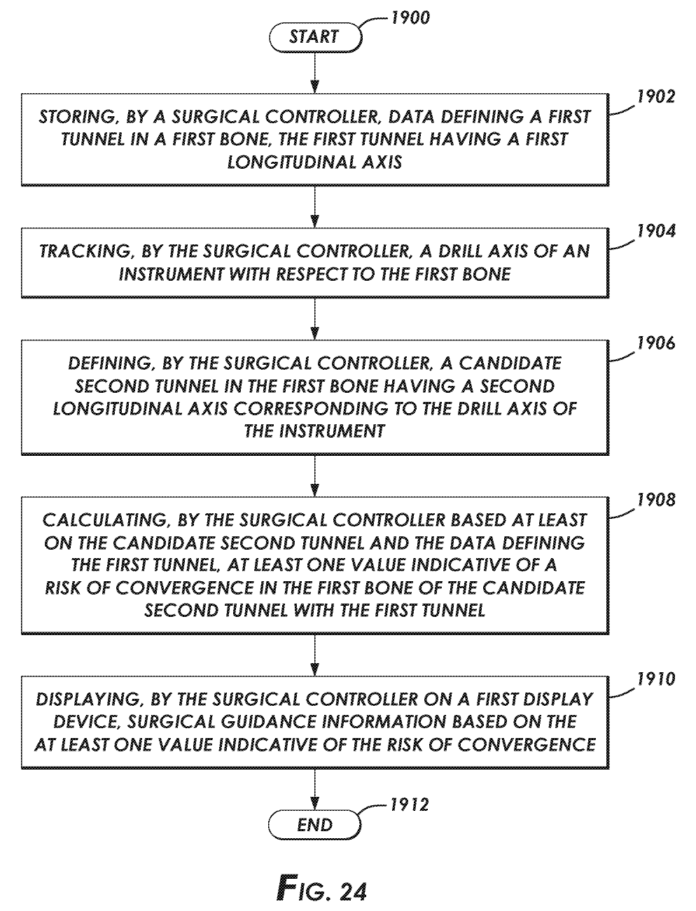

FIG. 24 shows a method in accordance with at least some embodiments. In particular, the method starts (block 1900) and comprises: storing, by a surgical controller, data defining a first tunnel in a first bone, the first tunnel having a first longitudinal axis (block 1902); tracking, by the surgical controller, a drill axis of an instrument with respect to the first bone (block 1904); defining, by the surgical controller, a candidate second tunnel in the first bone having a second longitudinal axis corresponding to the drill axis of the instrument (block 1906); calculating, by the surgical controller based at least on the candidate second tunnel and the data defining the first tunnel, at least one value indicative of a risk of convergence in the first bone of the candidate second tunnel with the first tunnel (block 1908); displaying, by the surgical controller on a first display device, surgical guidance information based on the at least one value indicative of the risk of convergence (block 1910). Thereafter, the method ends (block 1912). The example method may be implemented by computer instructions executed with the at least one processor of a computer system, such as the surgical controller 418 (FIG. 4).

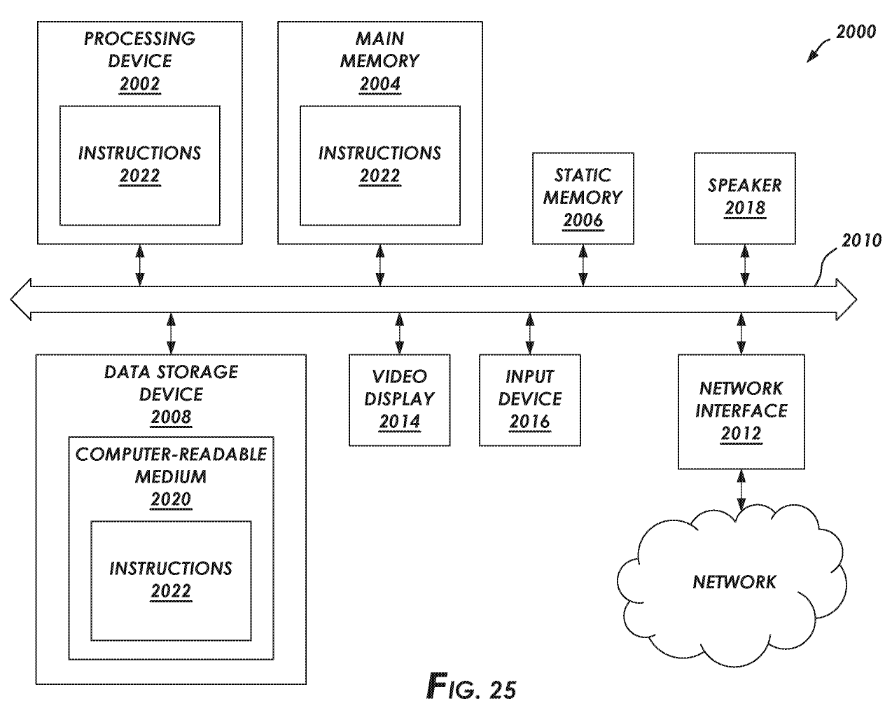

FIG. 25 shows an example computer system 2000. In one example, computer system 2000 may correspond to the surgical controller 418, a tablet device within the surgical room, or any other system that implements any or all the various methods discussed in this specification. The computer system 2000 may be connected (e.g., networked) to other computer systems in a local-area network (LAN), an intranet, and/or an extranet (e.g., device cart 402 network), or at certain times the Internet (e.g., when not in use in a surgical procedure). The computer system 2000 may be a server, a personal computer (PC), a tablet computer or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 2000 includes a processing device 2002, a main memory 2004 (e.g., read-only memory (ROM), flash memory, dynamic random access memory (DRAM) such as synchronous DRAM (SDRAM)), a static memory 2006 (e.g., flash memory, static random access memory (SRAM)), and a data storage device 2008, which communicate with each other via a bus 2010.

Processing device 2002 represents one or more general-purpose processing devices such as a microprocessor, central processing unit, or the like. More particularly, the processing device 2002 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 2002 may also be one or more special-purpose processing devices such as an application specific integrated circuit (ASIC), a field programmable gate array (FPGA), a digital signal processor (DSP), network processor, or the like. The processing device 2002 is configured to execute instructions for performing any of the operations and steps discussed herein. Once programmed with specific instructions, the processing device 2002, and thus the entire computer system 2000, becomes a special-purpose device, such as the surgical controller 418.

The computer system 2000 may further include a network interface device 2012 for communicating with any suitable network (e.g., the device cart 402 network). The computer system 2000 also may include a video display 2014 (e.g., display device 414), one or more input devices 2016 (e.g., a microphone, a keyboard, and/or a mouse), and one or more speakers 2018. In one illustrative example, the video display 2014 and the input device(s) 2016 may be combined into a single component or device (e.g., an LCD – liquid crystal display - touch screen).

The data storage device 2008 may include a computer-readable storage medium 2020 serving as memory on which the instructions 2022 (e.g., implementing any methods and any functions performed by any device and/or component depicted described herein) embodying any one or more of the methodologies or functions described herein is stored. The instructions 2022 may also reside, completely or at least partially, within the main memory 2004 and/or within the processing device 2002 during execution thereof by the computer system 2000. As such, the main memory 2004 and the processing device 2002 also constitute computer-readable media. In certain cases, the instructions 2022 may further be transmitted or received over a network via the network interface device 2012.

While the computer-readable storage medium 2020 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

For example, whereas in examples described the full replacement ligament length may be displayed on a display device based on a calculation by the surgical controller of the femoral tunnel length, the inter-articular ligament traversal distance, and the tibial length being navigated, alternatives are possible in which the information displayed during navigation includes only a determined tibial length, leaving the calculation of the overall ligament length to the surgeon rather. Various display options are possible.

In addition, depending on the kind/geometry of replacement ligament fixation device and/or other factors understood by a surgeon in a given procedure, a femoral or tibial tunnel length itself may not, alone, be determinative of the extent of actual femoral or tibial fixation of a replacement ligament. That is, of how much of the ligament is actually taken up in the tunnel. The surgical controller 418 may present the surgeon with an option to – during the femoral portion of a surgical procedure – manually enter an extent of femoral fixation – generally, a length amount - using a user interface element so that this can be stored by the surgical controller 418 and retrieved for use in guidance during a tibial portion of the surgical procedure. Alternatively or in some combination, the amount of fixation may be estimated by the surgical controller 418 based on information provided to the surgical controller 418 about the kind of fixation device to be used and/or other factors such as other measurements made manually and entered, and/or calculated. Alternatively or in some combination, the surgical controller 418 may present the surgeon with the option of manually providing such a fixation extent using a user interface element during the tibial portion of the surgical procedure rather than relying on either manually entered or calculated data during the femoral portion of the procedure, so that guidance provided by the surgical controller 418 during the tibial portion of the procedure can appropriately factor the fixation extent constraint. Furthermore, in examples, even if data is automatically estimated or entered by a surgeon during the femoral portion of the procedure, the surgical controller 418 may be configured to enable to surgeon to override the estimate/entry during the tibial portion of the procedure should the surgeon deem it appropriate to do so, so that the overriding information itself is used by the surgical controller 418 for the surgical guidance during that tibial portion of the procedure. It may be useful to provide the surgeon with various options that enable the surgeon to choose how and whether the surgical controller 418 provides surgical guidance, so that the surgeon can be provided with as much or as little surgical guidance as the surgeon would like for the particular procedure or stage. Various alternatives are possible.

Furthermore, while in examples various options have been presented in various display screens, it is to be understood that in other examples, for the sake of reducing clutter or confusion, or for improving usability, certain options and/or user interface elements may be made accessible only through interactions first with one or more configuration menus.

### Clauses

Clause 1. A method comprising:

storing, by a surgical controller, data defining a first tunnel in a first bone of a joint, the first tunnel having a first longitudinal axis;

tracking, by the surgical controller, a drill axis of an instrument with respect to a second bone of the joint;

defining, by the surgical controller, a candidate second tunnel in the second bone having a second longitudinal axis corresponding to the drill axis of the instrument;

calculating, by the surgical controller based at least on the candidate second tunnel and the data defining the first tunnel, at least one value indicative of a compatibility of the candidate second tunnel with the first tunnel for together supporting a replacement ligament; and

displaying, by the surgical controller on a first display device, surgical guidance information based on the at least one value indicative of the compatibility.

Clause 2. The method of clause 1, wherein calculating the at least one value indicative of the compatibility of the candidate second tunnel with the first tunnel comprises:

determining a first length based on or obtained from the data defining the first tunnel, the first length being the length of the first tunnel in the first bone;

calculating a second length, the second length being the length of the candidate second tunnel in the second bone;

calculating a third length, the third length being a distance between openings of the first tunnel in the first bone and the candidate second tunnel in the second bone;

calculating, as a candidate traversal distance, a sum of the first length, the second length, and the third length; and

determining, as the at least one value indicative of the compatibility, an amount of difference between the candidate traversal distance and a length of the replacement ligament.

Clause 3. The method of clause 2, wherein the surgical guidance information displayed on the first display device comprises:

a length value corresponding to the amount of difference between the candidate traversal distance and the length of the replacement ligament.

Clause 4. The method of clause 2, wherein the displaying, by the surgical controller on the first display device, the surgical guidance information comprises:

displaying an alert responsive to the candidate traversal distance being greater than the length of the replacement ligament; and

removing a previously-displayed alert responsive to the candidate traversal distance being less than or equal to the length of the replacement ligament.

Clause 5. The method of clause 4, further comprising:

during the tracking, displaying, by the surgical controller on the first display device, a representation of the candidate second tunnel in association with video frames of the second bone,

wherein the alert comprises a change in appearance of the representation.

Clause 6. The method of clause 5, wherein the change in appearance of the representation comprises at least one of a change in color of the representation, and a change in opacity of the representation.

Clause 7. The method of clause 1, wherein calculating the at least one value indicative of the compatibility of the candidate second tunnel with the first tunnel comprises:

generating, based at least on openings into the joint of the first tunnel in the first bone and the candidate second tunnel in the second bone, a candidate path of the replacement ligament through the joint; and

determining, as the at least one value indicative of the compatibility, an amount of impingement of the candidate path by at least one of the first bone and the second bone.

Clause 8. The method of clause 7, wherein the surgical guidance information displayed on the first display device comprises:

a percentage amount corresponding to the amount of impingement of the candidate path.

Clause 9. The method of clause 7, wherein the displaying, by the surgical controller on the first display device, the surgical guidance information comprises:

displaying an alert responsive to the amount of impingement being greater than or equal to a threshold level of impingement; and

removing a previously-displayed alert responsive to the impingement being less than the threshold level of impingement.

Clause 10. The method of clause 9, further comprising:

during the tracking, displaying, by the surgical controller on the first display device, a representation of the candidate second tunnel in association with video frames of the second bone,

wherein the alert comprises a change in appearance of the representation.

Clause 11. The method of clause 10, wherein the change in appearance of the representation comprises at least one of a change in color of the representation, and a change in opacity of the representation.

Clause 12. The method of clause 1, wherein the instrument is an adjustable aimer and the tracking, by the surgical controller, of the drill axis of the instrument with respect to the second bone of the joint comprises:

tracking a pose of the adjustable aimer with respect to the second bone;

receiving an aimer angle of the adjustable aimer; and

based on a predetermined geometry of the adjustable aimer, the pose of the adjustable aimer and the aimer angle, calculating the drill axis of the adjustable aimer with respect to the second bone.

Clause 13. The method of clause 12, comprising:

receiving, by the surgical controller, a user selection of the predetermined geometry of the adjustable aimer.

Clause 14. The method of clause 12, comprising:

processing, by the surgical controller, a machine-readable marker associated with the adjustable aimer to determine an identification of the adjustable aimer; and

retrieving, by the surgical controller, the predetermined geometry based on the identification.

Clause 15. The method of clause 12, wherein receiving the aimer angle of the adjustable aimer comprises:

processing, by the surgical controller, video frames of the adjustable aimer to determine the aimer angle.

Clause 16. The method of clause 1, wherein a 3D bone model is registered to the second bone.

Clause 17. The method of clause 16, further comprising:

displaying, by the surgical controller on the first display device and/or a second display device, a visual representation of the 3D bone model; and

displaying, by the surgical controller on the first display device and/or the second display device, at least the candidate second tunnel with respect to the visual representation of the 3D bone model.

Clause 18. The method of clause 17, wherein the displaying of the candidate second tunnel with respect to the visual representation of the 3D bone model is conducted by the surgical controller responsive to the surgical controller receiving, during the tracking, a selection by a user of a current pose of the instrument.

Clause 19. A surgical controller comprising:

at least one processor configured to couple to at least a first display device;

a memory coupled to the at least one processor, the memory storing instructions that, when executed by the at least one processor, cause the at least one processor to:

store data defining a first tunnel in a first bone of a joint, the first tunnel having a first longitudinal axis;

track a drill axis of an instrument with respect to a second bone of the joint;

define a candidate second tunnel in the second bone having a second longitudinal axis corresponding to the drill axis of the instrument;

calculate, based at least on the candidate second tunnel and the data defining the first tunnel, at least one value indicative of a compatibility of the candidate second tunnel with the first tunnel for together supporting a replacement ligament; and

display, on the first display device, surgical guidance information based on the at least one value indicative of the compatibility.

Clause 20. The surgical controller of clause 19, wherein to calculate the at least one value indicative of the compatibility of the candidate second tunnel with the first tunnel, the instructions cause the at least one processor to:

determine a first length based on or obtained from the data defining the first tunnel, the first length being the length of the first tunnel in the first bone;

calculate a second length, the second length being the length of the candidate second tunnel in the second bone;

calculate a third length, the third length being a distance between openings of the first tunnel in the first bone and the candidate second tunnel in the second bone;

calculate, as a candidate traversal distance, a sum of the first length, the second length, and the third length; and

determine, as the at least one value indicative of the compatibility, an amount of difference between the candidate traversal distance and a length of the replacement ligament.

Clause 21. The surgical controller of clause 20, wherein the surgical guidance information displayed on the first display device comprises:

a length value corresponding to the amount of difference between the candidate traversal distance and the length of the replacement ligament.

Clause 22. The surgical controller of clause 20, wherein for displaying, on the first display device, the surgical guidance information, the instructions cause the at least one processor to:

display an alert responsive to the candidate traversal distance being greater than the length of the replacement ligament; and

remove a previously-displayed alert responsive to the candidate traversal distance being less than or equal to the length of the replacement ligament.

Clause 23. The surgical controller of clause 22, wherein the instructions cause the at least one processor to:

during tracking of the drill axis, display, on the first display device, a representation of the candidate second tunnel in association with video frames of the second bone,

wherein the alert comprises a change in appearance of the representation.

Clause 24. The surgical controller of clause 23, wherein the change in appearance of the representation comprises at least one of a change in color of the representation, and a change in opacity of the representation.

Clause 25. The surgical controller of clause 19, wherein to calculate the at least one value indicative of the compatibility of the candidate second tunnel with the first tunnel, the instructions cause the at least one processor to:

generate, based at least on openings into the joint of the first tunnel in the first bone and the candidate second tunnel in the second bone, a candidate path of the replacement ligament through the joint; and

determine, as the at least one value indicative of the compatibility, an amount of impingement of the candidate path by at least one of the first bone and the second bone.

Clause 26. The surgical controller of clause 25, wherein the surgical guidance information displayed on the first display device comprises:

a percentage amount corresponding to the amount of impingement of the candidate path.

Clause 27. The surgical controller of clause 25, wherein to display, on the first display device, the surgical guidance information, the instructions cause the at least one processor to:

display an alert responsive to the amount of impingement being greater than or equal to a threshold level of impingement; and

remove a previously-displayed alert responsive to the impingement being less than the threshold level of impingement.

Clause 28. The surgical controller of clause 27, wherein the instructions cause the at least one processor to:

during tracking, display, on the first display device, a representation of the candidate second tunnel in association with video frames of the second bone,

wherein the alert comprises a change in appearance of the representation.

Clause 29. The surgical controller of clause 28, wherein the change in appearance of the representation comprises at least one of a change in color of the representation, and a change in opacity of the representation.

Clause 30. The surgical controller of clause 19, wherein the instrument is an adjustable aimer and, to track of the drill axis of the instrument with respect to the second bone of the joint the instructions cause the at least one processor to:

track a pose of the adjustable aimer with respect to the second bone;

receive an aimer angle of the adjustable aimer; and

based on a predetermined geometry of the adjustable aimer, the pose of the adjustable aimer and the aimer angle, calculate the drill axis of the adjustable aimer with respect to the second bone.

Clause 31. The surgical controller of clause 30, wherein the instructions cause the at least one processor to:

receive, by the surgical controller, a user selection of the predetermined geometry of the adjustable aimer.

Clause 32. The surgical controller of clause 30, wherein the instructions cause the at least one processor to:

process a machine-readable marker associated with the adjustable aimer to determine an identification of the adjustable aimer; and

retrieve the predetermined geometry based on the identification.

Clause 33. The surgical controller of clause 30, wherein to receive the aimer angle of the adjustable aimer the instructions cause the at least one processor to:

process video frames of the adjustable aimer to determine the aimer angle.

Clause 34. The surgical controller of clause 19, wherein a 3D bone model is registered to the second bone.

Clause 35. The surgical controller of clause 34, wherein the instructions cause the at least one processor to:

display, on the first display device and/or a second display device, a visual representation of the 3D bone model; and

Clause 37. A method comprising:

display at least the candidate second tunnel with respect to the visual representation of the 3D bone model.

Clause 36. The surgical controller of clause 35, wherein the instructions for causing the at least one processor to display the candidate second tunnel with respect to the visual representation of the 3D bone model are executed responsive to the surgical controller receiving, during tracking, a selection by a user of a current pose of the instrument.

storing, by a surgical controller, data defining a first tunnel in a first bone, the first tunnel having a first longitudinal axis;

tracking, by the surgical controller, a drill axis of an instrument with respect to the first bone;

defining, by the surgical controller, a candidate second tunnel in the first bone having a second longitudinal axis corresponding to the drill axis of the instrument;

calculating, by the surgical controller based at least on the candidate second tunnel and the data defining the first tunnel, at least one value indicative of a risk of convergence in the first bone of the candidate second tunnel with the first tunnel; and

displaying, by the surgical controller on a first display device, surgical guidance information based on the at least one value indicative of the risk of convergence.

Clause 38. The method of clause 37, wherein the calculating, by the surgical controller, the at least one value indicative of the risk of convergence of the candidate second tunnel with the first tunnel in the first bone comprises:

measuring based at least on the data defining the first tunnel, as the at least one value indicative of the risk of convergence, a distance between the closest points along the first tunnel and the candidate second tunnel within the first bone.

Clause 39. The method of clause 38, wherein the surgical guidance information displayed on the first display device comprises:

a bone bridge thickness amount corresponding to the distance between the closest points.

Clause 40. The method of clause 38, wherein the displaying, by the surgical controller on the first display device, the surgical guidance information comprises:

displaying an alert responsive to the distance between the closest points being below a threshold distance; and

removing a previously-displayed alert responsive to the distance between the closest points being greater than or equal to the threshold distance.

Clause 41. The method of clause 40, further comprising:

during the tracking, displaying, by the surgical controller on the first display device, a representation of the candidate second tunnel in association with video frames of the first bone,

wherein the alert comprises a change in appearance of the representation.

Clause 42. The method of clause 41, wherein the change in appearance of the representation comprises at least one of: a change in color of the representation, and a change in opacity of the representation.

Clause 43. The method of clause 41, further comprising:

displaying, by the surgical controller on the first display device, a representation of the first tunnel in association with the video frames of the first bone.

Clause 44. The method of clause 37, wherein a 3D bone model is registered to the first bone.

Clause 45. The method of clause 44, further comprising:

displaying, by the surgical controller on the first display device and/or a second display device, a visual representation of the 3D bone model; and

displaying, by the surgical controller on the first display device and/or the second display device, at least the first tunnel with respect to the visual representation of the 3D bone model.

Clause 46. The method of clause 45, comprising:

displaying, by the surgical controller on the first display device and/or the second display device, the candidate second tunnel with respect to the visual representation of the 3D bone model.

Clause 47. The method of clause 46, wherein the displaying of the candidate second tunnel with respect to the visual representation of the 3D bone model is conducted by the surgical controller responsive to the surgical controller receiving, during the tracking, a selection by a user of a current pose of the instrument.

Clause 48. A surgical controller comprising:

at least one processor configured to couple to at least a first display device;

a memory coupled to the at least one processor, the memory storing instructions that, when executed by the at least one processor, cause the at least one processor to:

store data defining a first tunnel in a first bone, the first tunnel having a first longitudinal axis;

track a drill axis of an instrument with respect to the first bone;

define a candidate second tunnel in the first bone having a second longitudinal axis corresponding to the drill axis of the instrument;

calculate, based at least on the candidate second tunnel and the data defining the first tunnel, at least one value indicative of a risk of convergence in the first bone of the candidate second tunnel with the first tunnel; and

display, on the first display device, surgical guidance information based on the at least one value indicative of the risk of convergence.

Clause 49. The surgical controller of clause 48, wherein to calculate the at least one value indicative of the risk of convergence of the candidate second tunnel with the first tunnel in the first bone, the instructions cause the at least one processor to:

measure based at least on the data defining the first tunnel, as the at least one value indicative of the risk of convergence, a distance between the closest points along the first tunnel and the candidate second tunnel within the first bone.

Clause 50. The surgical controller of clause 49, wherein the surgical guidance information displayed on the first display device comprises:

a bone bridge thickness amount corresponding to the distance between the closest points.

Clause 51. The surgical controller of clause 49, wherein to display, on the first display device, the surgical guidance information, the instructions cause the at least one processor to:

display an alert responsive to the distance between the closest points being below a threshold distance; and

remove a previously-displayed alert responsive to the distance between the closest points being greater than or equal to the threshold distance.

Clause 52. The surgical controller of clause 51, wherein the instructions cause the at least one processor to:

during tracking, display, on the first display device, a representation of the candidate second tunnel in association with video frames of the first bone,

wherein the alert comprises a change in appearance of the representation.

Clause 53. The surgical controller of clause 52, wherein the change in appearance of the representation comprises at least one of: a change in color of the representation, and a change in opacity of the representation.

Clause 54. The surgical controller of clause 52, wherein the instructions cause the at least one processor to:

display, on the first display device, a representation of the first tunnel in association with the video frames of the first bone.

Clause 55. The surgical controller of clause 48, wherein a 3D bone model is registered to the first bone.

Clause 56. The surgical controller of clause 55, wherein the instructions cause the at least one processor to:

display, on the first display device and/or a second display device, a visual representation of the 3D bone model; and

display at least the first tunnel with respect to the visual representation of the 3D bone model.

Clause 57. The surgical controller of clause 56, wherein the instructions cause the at least one processor to:

display, on the first display device and/or the second display device, the candidate second tunnel with respect to the visual representation of the 3D bone model.

Clause 58. The surgical controller of clause 57, wherein the instructions to cause the at least one processor to display the candidate second tunnel with respect to the visual representation of the 3D bone model are executed responsive to the surgical controller receiving, during tracking, a selection by a user of a current pose of the instrument.

What is claimed is:

1. A method comprising:
storing, by a surgical controller, data defining a first tunnel in a first bone of a joint, the first tunnel having a first longitudinal axis;
tracking, by the surgical controller, a drill axis of an instrument with respect to a second bone of the joint;
defining, by the surgical controller, a candidate second tunnel in the second bone having a second longitudinal axis corresponding to the drill axis of the instrument;
calculating, by the surgical controller based at least on the candidate second tunnel and the data defining the first tunnel, at least one value indicative of a compatibility of the candidate second tunnel with the first tunnel for together supporting a replacement ligament; and
displaying, by the surgical controller on a first display device, surgical guidance information based on the at least one value indicative of the compatibility.

2. The method of claim 1, wherein calculating the at least one value indicative of the compatibility of the candidate second tunnel with the first tunnel comprises:
determining a first length based on or obtained from the data defining the first tunnel, the first length being the length of the first tunnel in the first bone;
calculating a second length, the second length being the length of the candidate second tunnel in the second bone;
calculating a third length, the third length being a distance between openings of the first tunnel in the first bone and the candidate second tunnel in the second bone;
calculating, as a candidate traversal distance, a sum of the first length, the second length, and the third length; and
determining, as the at least one value indicative of the compatibility, an amount of difference between the candidate traversal distance and a length of the replacement ligament.

3. The method of claim 1, wherein calculating the at least one value indicative of the compatibility of the candidate second tunnel with the first tunnel comprises:
generating, based at least on openings into the joint of the first tunnel in the first bone and the candidate second tunnel in the second bone, a candidate path of the replacement ligament through the joint; and
determining, as the at least one value indicative of the compatibility, an amount of impingement of the candidate path by at least one of the first bone and the second bone.

4. The method of claim 1, wherein the instrument is an adjustable aimer and the tracking, by the surgical controller, of the drill axis of the instrument with respect to the second bone of the joint comprises:
tracking a pose of the adjustable aimer with respect to the second bone;
receiving an aimer angle of the adjustable aimer; and
based on a predetermined geometry of the adjustable aimer, the pose of the adjustable aimer and the aimer angle, calculating the drill axis of the adjustable aimer with respect to the second bone.

5. The method of claim 4, comprising:
receiving, by the surgical controller, a user selection of the predetermined geometry of the adjustable aimer.

6. The method of claim 4, comprising:
processing, by the surgical controller, a machine-readable marker associated with the adjustable aimer to determine an identification of the adjustable aimer; and
retrieving, by the surgical controller, the predetermined geometry based on the identification.

7. The method of claim 4, wherein receiving the aimer angle of the adjustable aimer comprises:
processing, by the surgical controller, video frames of the adjustable aimer to determine the aimer angle.

8. The method of claim 1, wherein a 3D bone model is registered to the second bone.

9. The method of claim 8, further comprising:
displaying, by the surgical controller on the first display device and/or a second display device, a visual representation of the 3D bone model; and
displaying, by the surgical controller on the first display device and/or the second display device, at least the candidate second tunnel with respect to the visual representation of the 3D bone model.

10. The method of claim 9, wherein the displaying of the candidate second tunnel with respect to the visual representation of the 3D bone model is conducted by the surgical controller responsive to the surgical controller receiving, during the tracking, a selection by a user of a current pose of the instrument.

11. A surgical controller comprising:
at least one processor configured to couple to at least a first display device;
a memory coupled to the at least one processor, the memory storing instructions that, when executed by the at least one processor, cause the at least one processor to:
store data defining a first tunnel in a first bone of a joint, the first tunnel having a first longitudinal axis;
track a drill axis of an instrument with respect to a second bone of the joint;
define a candidate second tunnel in the second bone having a second longitudinal axis corresponding to the drill axis of the instrument;
calculate, based at least on the candidate second tunnel and the data defining the first tunnel, at least one value indicative of a compatibility of the candidate second tunnel with the first tunnel for together supporting a replacement ligament; and
display, on the first display device, surgical guidance information based on the at least one value indicative of the compatibility.

12. The surgical controller of claim 11, wherein to calculate the at least one value indicative of the compatibility of the candidate second tunnel with the first tunnel, the instructions cause the at least one processor to:
determine a first length based on or obtained from the data defining the first tunnel, the first length being the length of the first tunnel in the first bone;
calculate a second length, the second length being the length of the candidate second tunnel in the second bone;
calculate a third length, the third length being a distance between openings of the first tunnel in the first bone and the candidate second tunnel in the second bone;
calculate, as a candidate traversal distance, a sum of the first length, the second length, and the third length; and
determine, as the at least one value indicative of the compatibility, an amount of difference between the candidate traversal distance and a length of the replacement ligament.

13. The surgical controller of claim 11, wherein to calculate the at least one value indicative of the compatibility of the candidate second tunnel with the first tunnel, the instructions cause the at least one processor to:
generate, based at least on openings into the joint of the first tunnel in the first bone and the candidate second tunnel in the second bone, a candidate path of the replacement ligament through the joint; and
determine, as the at least one value indicative of the compatibility, an amount of impingement of the candidate path by at least one of the first bone and the second bone.

14. The surgical controller of claim 11, wherein the instrument is an adjustable aimer and, to track of the drill axis of the instrument with respect to the second bone of the joint the instructions cause the at least one processor to:
track a pose of the adjustable aimer with respect to the second bone;
receive an aimer angle of the adjustable aimer; and
based on a predetermined geometry of the adjustable aimer, the pose of the adjustable aimer and the aimer angle, calculate the drill axis of the adjustable aimer with respect to the second bone.

15. The surgical controller of claim 14, wherein the instructions cause the at least one processor to:
receive, by the surgical controller, a user selection of the predetermined geometry of the adjustable aimer.

16. The surgical controller of claim 14, wherein the instructions cause the at least one processor to:
process a machine-readable marker associated with the adjustable aimer to determine an identification of the adjustable aimer; and
retrieve the predetermined geometry based on the identification.

17. The surgical controller of claim 14, wherein to receive the aimer angle of the adjustable aimer the instructions cause the at least one processor to:
process video frames of the adjustable aimer to determine the aimer angle.

18. The surgical controller of claim 11, wherein a 3D bone model is registered to the second bone.

19. The surgical controller of claim 18, wherein the instructions cause the at least one processor to:
display, on the first display device and/or a second display device, a visual representation of the 3D bone model; and
display at least the candidate second tunnel with respect to the visual representation of the 3D bone model.

20. The surgical controller of claim 19, wherein the instructions for causing the at least one processor to display the candidate second tunnel with respect to the visual representation of the 3D bone model are executed responsive to the surgical controller receiving, during tracking, a selection by a user of a current pose of the instrument.
